(19) United States
(12) Patent Application Publication
(10) Pub. No.: US 2009/0253720 A1
(43) Pub. Date: Oct. 8, 2009

(54) ANTIFOLATE COMPOSITIONS

(75) Inventors: Michael J. Roberts, Charlotte, NC (US); Harish K. Pimplaskar, Charlotte, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Chelsea Therapeutics, Inc.

(21) Appl. No.: 12/419,739

(22) Filed: Apr. 7, 2009

Related U.S. Application Data
(60) Provisional application No. 61/042,994, filed on Apr. 7, 2008.
(60) Provisional application No. 61/042,998, filed on Apr. 7, 2008.

(51) Int. Cl. A61K 31/517; A61P 29/00; A61P 11/06; A61P 19/02

(52) U.S. Cl. 514/266.4

(57) ABSTRACT

The present invention provides pharmaceutical compositions comprising an antifolate compound. The composition exhibit improved bioavailability, and they particularly incorporate beneficial excipients that increase solubility and bioavailability, such as cyclodextrins or compounds formed of fatty acid esters of glycerol and polyethylene glycol esters. The pharmaceutical compositions are useful in the treatment of multiple conditions, including abnormal cell proliferation, inflammatory diseases, asthma, and arthritis.

### CROSS-REFERENCE TO RELATED APPLICATIONS

The present application claims priority to U.S. Provisional Patent Application No. 61/042,994, filed Apr. 7, 2008, and U.S. Provisional Patent Application No. 61/042,998, filed Apr. 7, 2008, both of which are incorporated herein by reference in their entirety.

### FIELD OF THE INVENTION

The present application is directed to pharmaceutical compositions comprising active compounds. More specifically, the pharmaceutical compositions comprise antifolate compounds.

### BACKGROUND

Folic acid is a water-soluble B vitamin known by the systematic name N-[4(2-amino-4-hydroxy-pteridine-6-ylmethylamino)-benzoyl]-L(+)-glutamic acid and having the structure provided below in Formula (1).

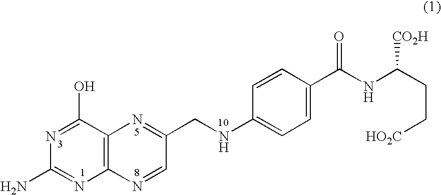

As seen in Formula (1), the folic acid structure can generally be described as being formed of a pteridine ring, a para-aminobenzoic acid moiety, and a glutamate moiety. Folic acid and its derivatives are necessary for metabolism and growth, particularly participating in the body's synthesis of thymidylate, amino acids, and purines. Derivatives of folic acid, such as naturally occurring folates, are known to have biochemical effects comparable to folic acid. Folic acid is known to be derivatized via hydrogenation, such as at the 1,4-diazine ring, or being methylated, formaldehydylated, or bridged, wherein substitution is generally at the N⁵ or N¹⁰ positions. Folates have been studied for efficacy in various uses including reduction in severity or incidence of birth defects, heart disease, stroke, memory loss, and age-related dementia.

Antifolate compounds, like folates, are structurally similar to folic acid; however, antifolate compounds function to disrupt folic acid metabolism. A review of antifolates is provided by Takamoto (1996) The Oncologist, 1:68-81, which is incorporated herein by reference. One specific group of antifolates, the so-called “classical antifolates,” is characterized by the presence of a folic acid p-aminobenzoylglutamic acid side chain, or a derivative of that side chain. Another group of antifolates, the so-called “nonclassical antifolates,” are characterized by the specific absence of the p-aminobenzoylglutamic group. Because antifolates have a physiological effect that is opposite the effect of folic acid, antifolates have been shown to exhibit useful physiological functions, such as the ability to destroy cancer cells by causing apoptosis.

Folate monoglutamylates and antifolate monoglutamylates are transported through cell membranes either in reduced form or unreduced form by carriers specific to those respective forms. Expression of these transport systems varies with cell type and cell growth conditions. After entering cells most folates, and many antifolates, are modified by polyglutamylation, wherein one glutamate residue is linked to a second glutamate residue at the α carboxy group via a peptide bond. This leads to formation of poly-L-γ-glutamylates, usually by addition of three to six glutamate residues. Enzymes that act on folates have a higher affinity for the polyglutamylated forms. Therefore, polyglutamylated folates generally exhibit a longer retention time within the cell.

An intact folate enzyme pathway is important to maintain de novo synthesis of the building blocks of DNA, as well as many important amino acids. Antifolate targets include the various enzymes involved in folate metabolism, including (i) dihydrofolate reductase (DHFR); (ii) thymidylate synthase (TS); (iii) folylpolyglutamyl synthase; and (iv) glycinamide ribonucleotide transformylase (GARFT) and aminoimidazole carboxamide ribonucleotide transformylase (AICART).

The reduced folate carrier (RFC), which is a transmembrane glycoprotein, plays an active role in the folate pathway transporting reduced folate into mammalian cells via the carrier mediated mechanism (as opposed to the receptor mediated mechanism). The RFC also transports antifolates, such as methotrexate. Thus, mediating the ability of RFC to function can affect the ability of cells to uptake reduced folates.

Polyglutamylated folates can function as enzyme cofactors, whereas polyglutamylated antifolates generally function as enzyme inhibitors. Moreover, interference with folate metabolism prevents de novo synthesis of DNA and some amino acids, thereby enabling antifolate selective cytotoxicity. Methotrexate, the structure of which is provided in Formula (2), is one antifolate that has shown use in cancer treatment, particularly treatment of acute leukemia, non-Hodgkin's lymphoma, breast cancer, head and neck cancer, choriocarcinoma, osteogenic sarcoma, and bladder cancer.

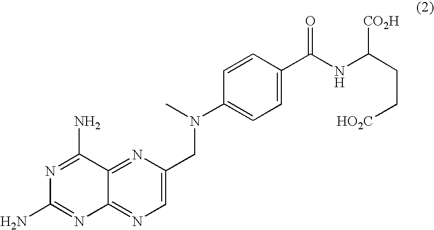

Nair et al. (J. Med. Chem. (1991) 34:222-227), incorporated herein by reference, demonstrated that polyglutamylation of classical antifolates was not essential for anti-tumor activity and may even be undesirable in that polyglutamylation can lead to a loss of drug pharmacological activity and target specificity. This was followed by the discovery of numerous nonpolyglutamylatable classical antifolates. See Nair et al. (1998) Proc. Amer. Assoc. Cancer Research 39:431, which is incorporated herein by reference. One particular group of nonpolyglutamylatable antifolates are characterized by a methylidene group (i.e., a ═CH₂ substituent) at the 4-position of the glutamate moiety. The presence of this chemical group has been shown to affect biological activity of the antifolate compound. See Nair et al. (1996) Cellular Pharmacology 3:29, which is incorporated herein by reference.

Further folic acid derivatives have also been studied in the search for antifolates with increased metabolic stability allowing for smaller doses and less frequent patient administration. For example, a dideaza (i.e., quinazoline-based) analog has been shown to avoid physiological hydroxylation on the pteridine ring system. Furthermore, replacement of the secondary amine nitrogen atom with an optionally substituted carbon atom has been shown to protect neighboring bonds from physiological cleavage.

One example of an antifolate having carbon replacement of the secondary amine nitrogen is 4-amino-4-deoxy-10-deazapteroyl-γ-methyleneglutamic acid—more commonly referred to as MDAM—the structure of which is provided in Formula (3).

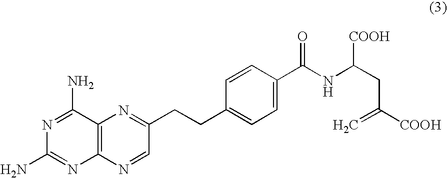

The L-enantiomer of MDAM has been shown to exhibit increased physiological activity. See U.S. Pat. No. 5,550,128, which is incorporated herein by reference. Another example of a classical antifolate designed for metabolic stability is ZD1694, which is shown in Formula (4).

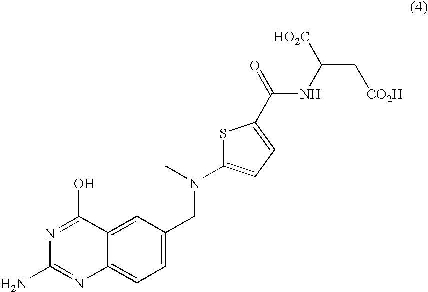

A group of antifolate compounds according to the structure shown in Formula (5) combines several of the molecular features described above, and this group of compounds is known by the names MobileTrexate, Mobile Trex, Mobiltrex, or M-Trex.

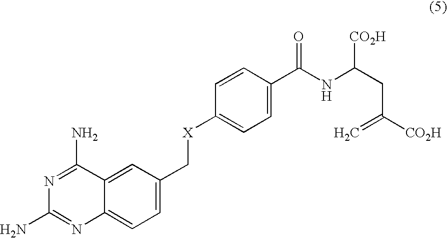

As shown in Formula (5), this group of compounds encompasses M-Trex, wherein X can be CH₂, CHCH₃, CH(CH₂CH₃), NH, or NCH₃.

The effectiveness of antifolates as pharmaceutical compounds arises from other factors in addition to metabolic inertness, as described above. The multiple enzymes involved in folic acid metabolism within the body present a choice of inhibition targets for antifolates. In other words, it is possible for antifolates to vary as to which enzyme(s) they inhibit. For example, some antifolates inhibit primarily dihydrofolate reductase (DHFR), while other antifolates inhibit primarily thymidylate synthase (TS), glycinamide ribonucleotide formyltranferase (GARFT), or aminoimidazole carboxamide ribonucleotide transformylase, while still other antifolates inhibit combinations of these enzymes.

In light of the usefulness of antifolates in treating a variety of conditions, there remains a need in the art for pharmaceutical compositions that can safely and effectively deliver the antifolates to a patient in need of treatment.

### SUMMARY OF THE INVENTION

The present invention provides pharmaceutical compositions comprising antifolate compounds. The pharmaceutical compositions provide the antifolate compounds in a form exhibiting excellent bioavailability. In specific embodiments, the antifolate compounds used in the compositions are in the form of salts. Such salts provide for improved solubility, particularly in lower pH ranges. The salt forms of the antifolate compounds are also beneficial for increasing the amount of the active compounds that is made available for biological activity when administered orally, even when the compositions comprise a reduced amount of the active antifolate compound. The pharmaceutical compositions of the invention are useful in the treatment of a variety of conditions including, but not limited to, abnormal cellular proliferation, asthma and other inflammatory diseases, and rheumatoid arthritis and other autoimmune diseases.

In one embodiment, the present invention is directed to a pharmaceutical composition comprising an antifolate compound according to Formula (6):

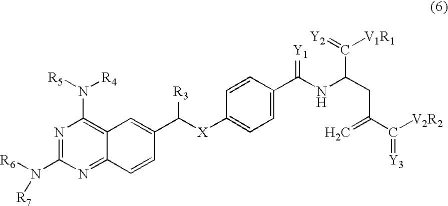

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof. In specific embodiments, the pharmaceutical composition further comprises an excipient that increases one or both of solubility and bioavailability of the antifolate compound. In particular, the excipient can comprise fatty acid esters of glycerol and polyethylene glycol esters and/or cyclodextrins. In certain embodiments, the excipient comprises GELUCIRE®, and particularly GELUCIRE® 44/14.

In other embodiments, the pharmaceutical composition of the invention comprises an antifolate compound according to formula (7):

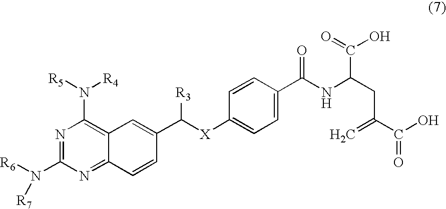

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof.

In still further embodiments, the pharmaceutical composition according to the invention comprises an antifolate compound according to Formula (9):

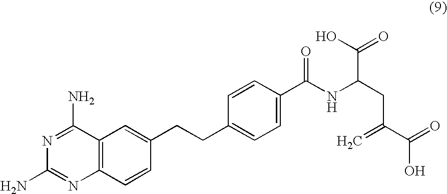

or a pharmaceutically acceptable ester, amide, salt, solvate, enantiomer, or prodrug thereof. In specific embodiments, the antifolate compound comprises a salt of the compound according to Formula (9), preferably an alkali metal salt of the compound, and particularly preferably a disodium salt or dipotassium salt of the compound according to Formula (9). In certain embodiments, the salt is in a crystalline form.

In other embodiments, it is beneficial for the pharmaceutical composition to comprise an antifolate compound that is in the (S) enantiomeric form. Preferably, the antifolate compound exhibits an enantiomeric purity for the (S) enantiomer of at least about 90%, more preferably at least about 95%, and still more preferably, at least about 99%. In one specific embodiment, the invention provides a pharmaceutical composition comprising an antifolate compound (such as the compound of Formula (9)), as a crystalline, disodium salt in the (S) enantiomeric form, the compound exhibiting an enantiomeric purity for the (S) enantiomer of at least about 99%.

In some embodiments, the invention particularly provides pharmaceutical compositions comprising an antifolate compound comprises a compound according to Formula (12):

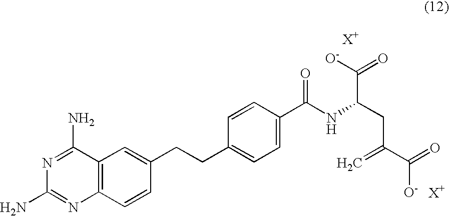

wherein each X⁺ independently is a salt-forming counterion, and wherein the antifolate compound is in the (S) enantiomeric form. More particularly, the antifolate compound may exhibit an enantiomeric purity for the (S) enantiomer of at least about 90%, at least about 95%, or at least about 99%. Further, the compound according to Formula (12) may be a crystalline, disodium salt in the (S) enantiomeric form exhibiting a defined enantiomeric purity for the (S) enantiomer (e.g., at least about 99%). Moreover, the compound according to Formula (12) may be a crystalline, dipotassium salt in the (S) enantiomeric form exhibiting a defined enantiomeric purity for the (S) enantiomer (e.g., at least about 99%).

In some embodiments, the pharmaceutical composition according to the invention may comprise further components. Non-limiting examples of such components include bulking agents (e.g., mannitol), lubricants (e.g., magnesium stearate), and anti-adherents (e.g., silicon dioxide).

In one embodiment, the invention provides a pharmaceutical composition comprising an alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%. The composition further may comprise an excipient that increases one or both of solubility and bioavailability of the alkali metal salt compound.

The invention also provides pharmaceutical compositions comprising further active agents. In particularly, the pharmaceutical composition can comprise one or more antifolate compounds as described herein in combination with one or more further active ingredients.

In further embodiments, the present invention also provides methods of treating various conditions. For example, in certain embodiments, the invention provides a method for treating a condition selected from the group consisting of abnormal cell proliferation, inflammation, asthma, and arthritis. Preferably, method comprising administering to a subject in need of treatment a pharmaceutical composition, such as described herein.

In still other embodiments, the invention provides methods of preparing pharmaceutical compositions. In on embodiment, the method is directed to preparing a pharmaceutical composition comprising an antifolate compound according to Formula (6):

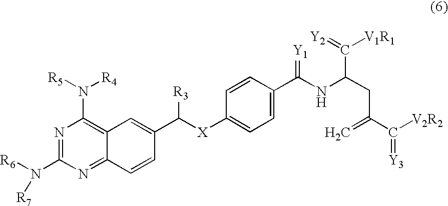

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof. Specifically, the method may comprise the following steps: forming a mixture of the antifolate compound, a molten polyglycolized glyceride, a first amount of a bulking agent, and a first amount of a lubricant; granulating the formed mixture; and combining the granulated mixture with a second amount of a bulking agent and a second amount of a lubricant.

### BRIEF DESCRIPTION OF THE DRAWINGS

Having thus described the invention in general terms, reference will now be made to the accompanying drawings, which are not necessarily drawn to scale, and wherein:

FIG. 1 is a graph of pH solubility for an antifolate compound useful in pharmaceutical compositions according to certain embodiments of the invention, the compound being in either the free acid form or the sodium salt form;

FIG. 2 is graph of comparative dissolution over time of an antifolate compound useful in pharmaceutical compositions according to certain embodiments of the invention, the compound being in either the free acid form or the sodium salt form;

FIG. 3 is a graph of comparative dissolution over time of an antifolate compound useful in pharmaceutical compositions according to certain embodiments of the invention, the compound being the free acid form of the compound alone, the sodium salt form of the compound alone, or the sodium salt form of the compound in a pharmaceutical composition including GELUCIRE® 44/14;

FIG. 4 is a graph of a comparative dissolution over time of an antifolate compound useful in pharmaceutical compositions according to certain embodiments of the invention, the compound being the free acid form of the compound alone, the sodium salt form of the compound alone, or the sodium salt form of the compound in a pharmaceutical composition including beta-cyclodextrin; and

FIG. 5 is an X-ray powder diffraction pattern graph of a salt compound useful in a pharmaceutical composition according to one embodiment of the invention.

### DETAILED DESCRIPTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The invention provides pharmaceutical compositions comprising antifolate compounds. These compounds can be used in the pharmaceutical composition either directly or in the form of their pharmaceutically active esters, amides, salts, solvates, or prodrugs. In preferred embodiments, the antifolate compounds are in the form of salts, particularly alkali metal salts. The pharmaceutical compositions provide increased activity and bioavailability, even at reduced dosing of the active antifolate compounds, and the pharmaceutical compositions are useful in the treatment of a number of conditions and diseases, particularly for the treatment of abnormal cell proliferation, inflammation, arthritis, or asthma.

### I. DEFINITIONS

The term “metabolically inert antifolate” as used herein means compounds that are (i) folic acid analogs capable of disrupting folate metabolism and (ii) non-polyglutamylatable. In certain embodiments, the term can mean compounds that are also (iii) non-hydroxylatable.

The term “alkali metal” as used herein means Group IA elements and particularly includes sodium, lithium, and potassium; the term “alkali metal salt” as used herein means an ionic compound wherein the cation moiety of the compound comprises an alkali metal, particularly sodium, lithium, or potassium.

The term “alkyl” as used herein means saturated straight, branched, or cyclic hydrocarbon groups. In particular embodiments, alkyl refers to groups comprising 1 to 10 carbon atoms (“C₁₋₁₀ alkyl”). In further embodiments, alkyl refers to groups comprising 1 to 8 carbon atoms (“C₁₋₈ alkyl”), 1 to 6 carbon atoms (“C₁₋₆ alkyl”), or 1 to 4 carbon atoms (“C₁₋₄ alkyl”). In specific embodiments, alkyl refers to methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. Substituted alkyl refers to alkyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkenyl” as used herein means alkyl moieties wherein at least one saturated C—C bond is replaced by a double bond. In particular embodiments, alkenyl refers to groups comprising 1 to 10 carbon atoms (“C₁₋₁₀ alkenyl”). In further embodiments, alkenyl refers to groups comprising 1 to 8 carbon atoms (“C₁₋₈ alkenyl”), 1 to 6 carbon atoms (“C₁₋₆ alkenyl”), or 1 to 4 carbon atoms (“C₁₋₄ alkenyl”). In specific embodiments, alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Substituted alkenyl refers to alkenyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkynyl” as used herein means alkynyl moieties wherein at least one saturated C—C bond is replaced by a triple bond. In particular embodiments, alkynyl refers to groups comprising 1 to 10 carbon atoms (“C₁₋₁₀ alkynyl”). In further embodiments, alkynyl refers to groups comprising 1 to 8 carbon atoms (“C₁₋₈ alkynyl”), 1 to 6 carbon atoms (“C₁₋₆ alkynyl”), or 1 to 4 carbon atoms (“C₁₋₄ alkynyl”). In specific embodiments, alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl. Substituted alkynyl refers to alkynyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C₁₋₁₀ alkoxy”). In further embodiments, alkoxy refers to oxygen-linked groups comprising 1 to 8 carbon atoms (“C₁₋₈ alkoxy”), 1 to 6 carbon atoms (“C₁₋₆ alkoxy”), or 1 to 4 carbon atoms (“C₁₋₄ alkoxy”). Substituted alkoxy refers to alkoxy substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “halo” or “halogen” as used herein means fluorine, chlorine, bromine, or iodine.

The term “aryl” as used herein means a stable monocyclic, bicyclic, or tricyclic carbon ring of up to 8 members in each ring, wherein at least one ring is aromatic as defined by the Hückel 4n+2 rule. Exemplary aryl groups according to the invention include phenyl, naphthyl, tetrahydronaphthyl, and biphenyl. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate.

The terms “aralkyl” and “arylalkyl” as used herein mean an aryl group as defined above linked to the molecule through an alkyl group as defined above.

The terms “alkaryl” and “alkylaryl” as used herein means an alkyl group as defined above linked to the molecule through an aryl group as defined above.

The term “acyl” as used herein means a carboxylic acid ester in which the non-carbonyl moiety of the ester group is selected from straight, branched, or cyclic alkyl or lower alkyl; alkoxyalkyl including methoxymethyl; aralkyl including benzyl; aryloxyalkyl such as phenoxymethyl; aryl including phenyl optionally substituted with halogen, C₁-C₆ alkyl or C₁-C₆ alkoxy; sulfonate esters such as alkyl or aralkyl sulphonyl including methanesulfonyl; mono-, di-, or triphosphate ester; trityl or monomethoxytrityl; substituted benzyl; trialkylsilyl such as dimethyl-t-butylsilyl or diphenylmethylsilyl. Aryl groups in the esters optimally comprise a phenyl group.

The term “amino” as used herein means a moiety represented by the structure NR₂, and includes primary amines, and secondary and tertiary amines substituted by alkyl (i.e., alkylamino). Thus, R₂ may represent two hydrogen atoms, two alkyl moieties, or one hydrogen atom and one alkyl moiety.

The terms “alkylamino” and “arylamino” as used herein mean an amino group that has one or two alkyl or aryl substituents, respectively.

The term “analogue” as used herein means a compound in which one or more individual atoms or functional groups have been replaced, either with a different atom or a different functional, generally giving rise to a compound with similar properties.

The term “derivative” as used herein means a compound that is formed from a similar, beginning compound by attaching another molecule or atom to the beginning compound. Further, derivatives, according to the invention, encompass one or more compounds formed from a precursor compound through addition of one or more atoms or molecules or through combining two or more precursor compounds.

The term “prodrug” as used herein means any compound which, when administered to a mammal, is converted in whole or in part to a compound of the invention.

The term “active metabolite” as used herein means a physiologically active compound which results from the metabolism of a compound of the invention, or a prodrug thereof, when such compound or prodrug is administered to a mammal.

The terms “therapeutically effective amount” or “therapeutically effective dose” as used herein are interchangeable and mean a concentration of a compound according to the invention, or a biologically active variant thereof, sufficient to elicit the desired therapeutic effect according to the methods of treatment described herein.

The term “pharmaceutically acceptable carrier” as used herein means a carrier that is conventionally used in the art to facilitate the storage, administration, and/or the healing effect of a biologically active agent.

The term “intermittent administration” as used herein means administration of a therapeutically effective dose of a composition according to the invention, followed by a time period of discontinuance, which is then followed by another administration of a therapeutically effective dose, and so forth.

The term “antiproliferative agent” as used herein means a compound that decreases the hyperproliferation of cells.

The term “abnormal cell proliferation” as used herein means a disease or condition characterized by the inappropriate growth or multiplication of one or more cell types relative to the growth of that cell type or types in an individual not suffering from that disease or condition.

The term “cancer” as used herein means a disease or condition characterized by uncontrolled, abnormal growth of cells, which can spread locally or through the bloodstream and lymphatic system to other parts of the body. The term includes tumor-forming or non-tumor forming cancers, and includes various types of cancers, such as primary tumors and tumor metastasis.

The term “tumor” as used herein means an abnormal mass of cells within a multicellular organism that results from excessive cell division that is uncontrolled and progressive, also called a neoplasm. A tumor may either be benign or malignant.

The term “fibrotic disorders” as used herein means fibrosis and other medical complications of fibrosis which result in whole or in part from the proliferation of fibroblasts.

The term “arthritis” as used herein means an inflammatory disorder affecting joints that can be infective, autoimmune, or traumatic in origin.

Chemical nomenclature using the symbols “D” and “L” or “R” and “S” are understood to relate the absolute configuration, or three-dimensional arrangement, of atoms or groups around a chiral element, which may be a center, usually an atom, an axis, or a plane. As used herein, the “D/L” system and the “R/S” systems are meant to be used interchangeably such that “D” in the former system corresponds to “R” in the later system and “L” in the former system corresponds to “S” in the later system.

### II. Compounds

The pharmaceutical compositions of the invention comprise one or more antifolate compounds. In specific embodiments, the antifolate compounds are metabolically inert antifolates. As recognized in the art, antifolates are compounds that interfere with various stages of folate metabolism. Thus, the compounds of the invention can particularly be used in pharmaceutical compositions useful for the treatment of diseases and conditions related to or capable of being treated by disruption of folate metabolism, or other biological mechanisms related to folate metabolism.

In one embodiment, the pharmaceutical compositions of the present invention comprise antifolate compounds having the structure provided in Formula (6),

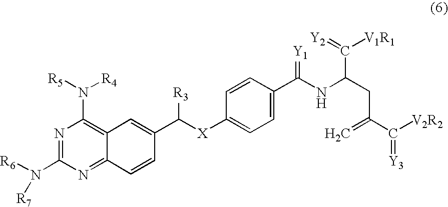

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In another embodiment, the pharmaceutical compositions of the present invention comprise compounds having the structure provided in Formula (7)

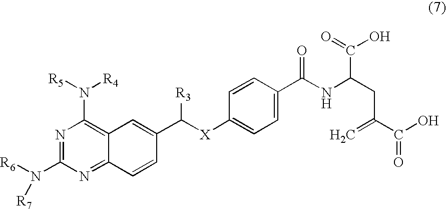

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₉ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In yet another embodiment, the pharmaceutical compositions of the present invention comprise antifolate compounds having the structure provided in Formula (8)

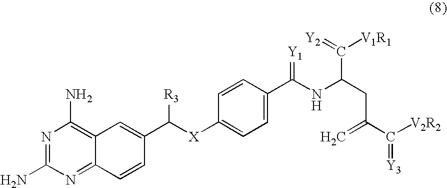

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₈ is H, optionally substituted alkyl, optionally substituted alkenyl, optionally substituted alkynyl, acyl, —C(O)-alkyl, —C(O)-alkenyl, or —C(O)-alkynyl as well as pharmaceutically acceptable esters, amides, salts, solvates, enantiomers, and prodrugs thereof.

In one particular embodiment, the present invention provides pharmaceutical compositions comprising an antifolate compound having the structure provided in Formula (9).

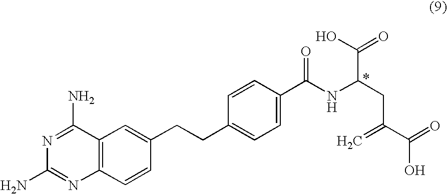

The compound of Formula (9) has been shown to have activity for the treatment of abnormal cellular proliferation, inflammation disorders, and autoimmune diseases. This compound may particularly be known by the name 2-{4-[2-(2,4-diamino-quinazolin-6-yl)-ethyl]-benzoylamino}-4-methylene-pentanedioic acid. The compound may also be known as gamma methylene glutamate 5,8,10-trideaza aminopterin or 5,8-dideaza MDAM. The antifolate compound of Formula (9) is non-polyglutamylatable, non-hydroxylatable, and capable of disrupting folate metabolism. The compound has also shown effectiveness in killing large numbers of human leukemia cells and human solid tumor cells in culture at therapeutically relevant concentrations, and has further shown activity as an anti-inflammatory agent in an animal model of asthma. Unfortunately, the compound suffers from low bioavailability, and the acid form exhibits low solubility, as further described below.

Biologically active variants of the compounds set forth above are particularly also encompassed by the invention. Such variants should retain the general biological activity of the original compounds; however, the presence of additional activities would not necessarily limit the use thereof in the present invention. Such activity may be evaluated using standard testing methods and bioassays recognizable by the skilled artisan in the field as generally being useful for identifying such activity.

According to one embodiment of the invention, suitable biologically active variants comprise one or more analogues or derivatives of the compounds described above. Indeed, a single compound, such as those described above, may give rise to an entire family of analogues or derivatives having similar activity and, therefore, usefulness according to the present invention. Likewise, a single compound, such as those described above, may represent a single family member of a greater class of compounds useful according to the present invention. Accordingly, the present invention fully encompasses not only the compounds described above, but analogues and derivatives of such compounds, particularly those identifiable by methods commonly known in the art and recognizable to the skilled artisan.

The compounds disclosed herein may contain chiral centers, which may be either of the (R) or (S) configuration, or may comprise a mixture thereof. Accordingly, the present invention also includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described herein.

The compound of Formula (9), in particular, is a chiral compound, the chiral center being indicated with an asterisk. Accordingly, the antifolate compound of Formula (9) can exist as two separate enantiomers—either the (R) enantiomer or the (S) enantiomer. Typically, the antifolate compound of Formula (9) exists as a racemic mixture of the two enantiomers.

Various methods are known in the art for preparing optically active forms and determining activity. Such methods include standard tests described herein and other similar tests which are well known in the art. Examples of methods that can be used to obtain optical isomers of the compounds useful according to the present invention include the following:

i) physical separation of crystals whereby macroscopic crystals of the individual enantiomers are manually separated. This technique may particularly be used when crystals of the separate enantiomers exist (i.e., the material is a conglomerate), and the crystals are visually distinct;

ii) simultaneous crystallization whereby the individual enantiomers are separately crystallized from a solution of the racemate, possible only if the latter is a conglomerate in the solid state;

iii) enzymatic resolutions whereby partial or complete separation of a racemate by virtue of differing rates of reaction for the enantiomers with an enzyme;

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis whereby the desired enantiomer is synthesized from an achiral precursor under conditions that produce asymmetry (i.e., chirality) in the product, which may be achieved using chiral catalysts or chiral auxiliaries;

vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;

viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors whereby the desired enantiomer is obtained from non-chiral starting materials and where the stereochemical integrity is not or is only minimally compromised over the course of the synthesis;

x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography whereby the racemate is volatilized and enantiomers are separated by virtue of their differing interactions in the gaseous mobile phase with a column containing a fixed non-racemic chiral adsorbent phase;

xii) extraction with chiral solvents whereby the enantiomers are separated by virtue of preferential dissolution of one enantiomer into a particular chiral solvent; and

xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In one embodiment, the pharmaceutical compositions of the invention comprise (S)-2-{4-[2-(2,4-diamino-quinazolin-6-yl)-ethyl]-benzoylamino}-4-methylene-pentanedioic acid, which is shown in Formula (10). The compound of Formula (10) is the (S) enantiomer of the compound shown in Formula (9). The (S) enantiomer is particularly useful in the pharmaceutical compositions of the invention in light of its increased activity in comparison to the (R) enantiomer. This is illustrated in the Examples appended hereto.

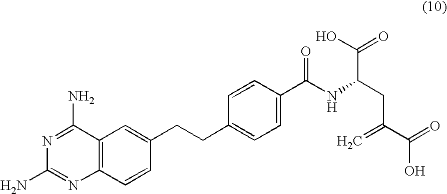

The antifolate compounds used in the inventive pharmaceutical compositions optionally may be provided in an enantiomerically enriched form, such as a mixture of enantiomers in which one enantiomer is present in excess (given as a mole fraction or a weight fraction). Enantiomeric excess is understood to exist where a chemical substance comprises two enantiomers of the same compound and one enantiomer is present in a greater amount than the other enantiomer. Unlike racemic mixtures, these mixtures will show a net optical rotation. With knowledge of the specific rotation of the mixture and the specific rotation of the pure enantiomer, the enantiomeric excess (abbreviated “ee”) can be determined by known methods. Direct determination of the quantities of each enantiomer present in the mixture (e.g., as a weight %) is possible with NMR spectroscopy and chiral column chromatography.

In one embodiment, the pharmaceutical compositions of the invention comprise a compound according to Formula (9), wherein the (S) enantiomer, as shown in Formula (10), is present in an enantiomeric excess. In such embodiments, the compositions can be referred to as comprising the compound of Formula (9) in an optically purified form in relation to the (S) enantiomer. Likewise, the compositions comprising an enantiomeric excess of the (S) enantiomer can be referred to as having a specific enantiomeric purity.

Preferably, the antifolate compounds used in the pharmaceutical compositions of the invention are enantiomerically pure for the (S) enantiomer such that greater than 50% of the compound present in the composition is the (S) enantiomer. In specific embodiments, the pharmaceutical compositions of the invention comprise an antifolate compound according to Formula (9) having an enantiomeric purity for the (S) enantiomer of at least about 75%. In other words, at least about 75% of the antifolate compound present in the composition is in the (S) form. In further embodiments, the antifolate compound of Formula (9) used in the inventive pharmaceutical compositions has an enantiomeric purity for the (S) enantiomer of at least about 80%, at least about 85%, at least about 90%, at least about 95%, at least about 96%, at least about 97%, at least about 98%, at least about 99%, at least about 99.5%, at least about 99.6%, at least about 99.7%, or at least about 99.8%.

The compounds described herein for use in the inventive pharmaceutical compositions can, in certain embodiments, be in the form of an ester, amide, salt, solvate, prodrug, or metabolite provided they maintain pharmacological activity according to the present invention. Esters, amides, salts, solvates, prodrugs, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

Examples of pharmaceutically acceptable salts of the compounds useful according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxalacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates. An acid addition salt may be reconverted to the free base by treatment with a suitable base.

If a compound of the invention is an acid, the desired salt may be prepared by any suitable method known to the art, including treatment of the free acid with an inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

If a compound useful according to the invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such a p-toluenesulfonic acid or ethanesulfonic acid, or the like.

Esters of the compounds according to the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Prodrugs are typically prepared by covalent attachment of a moiety, which results in a compound that is therapeutically inactive until modified by an individual's metabolic system. Examples of pharmaceutically acceptable solvates include, but are not limited to, compounds according to the invention in combination with water, isopropanol, ethanol, methanol, DMSO, ethyl acetate, acetic acid, or ethanolamine.

In particular embodiments, the antifolate compound used in the pharmaceutical compositions comprises a salt of the antifolate compounds described above. In preferred embodiments, the invention provides a pharmaceutical composition comprising a salt of the compound according to Formula (11).

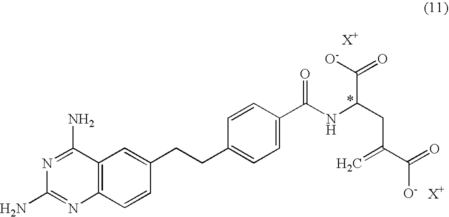

In Formula (11), the asterisk again denotes a chiral center, X⁺ can be any suitable salt-forming counterion, and each X⁺ can be the same or different. In specific embodiments, X⁺ is an alkali metal. In one preferred embodiment, X⁺ is a sodium cation. In another preferred embodiment, X⁺ is a potassium cation. In a specific embodiment, the composition of the invention comprises a disodium salt according to Formula (11). In still another specific embodiment, the composition of the invention comprises a dipotassium salt according to Formula (11). Of course, it is understood that other cationic moieties could be used as X⁺ in the compound of Formula (11). Moreover, the invention also encompasses salt forms according to Formula (11) that can be enantiomerically pure for the (R) enantiomer, enantiomerically pure for the (S) enantiomer, or in a racemic form. Such enantiomeric purity can be as previously described above.

Salts of antifolate compounds, such as the compounds of Formula (11), can be particularly useful in the pharmaceutical compositions of the invention in light of their favorable physico-chemical properties. Example 1 (appended hereto) describes a salt screen of the racemic free acid compound of Formula (9) using 19 different pharmaceutically acceptable acids and six bases.

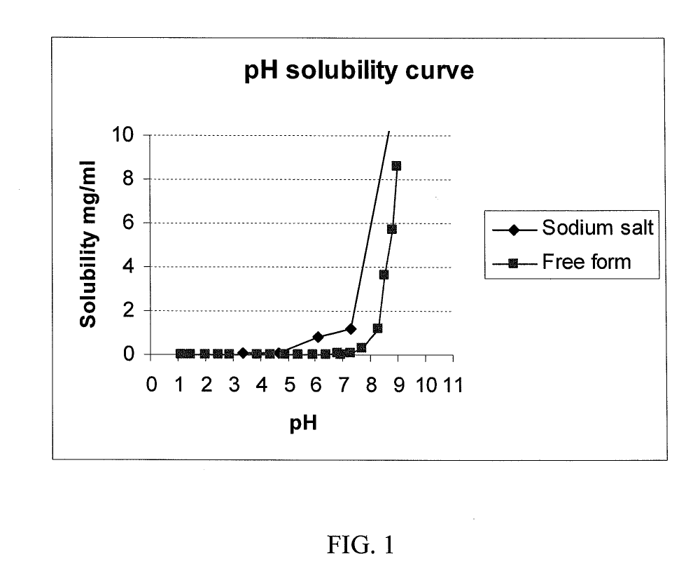

The disodium salt of the compound of Formula (11) has particularly been shown to have improved solubility characteristics in comparison to the dioic acid form, as shown in Formula (9). This is illustrated in FIG. 1 by the graph showing solubility as a function of pH. In FIG. 1, the “Free Form” refers to the antifolate compound according to Formula (9) and the “Sodium salt” refers to the disodium salt of the compound according to Formula (11). As seen in FIG. 1, the sodium salt of the antifolate compound exhibits greater solubility at a pH more closely relating to physiological pH.

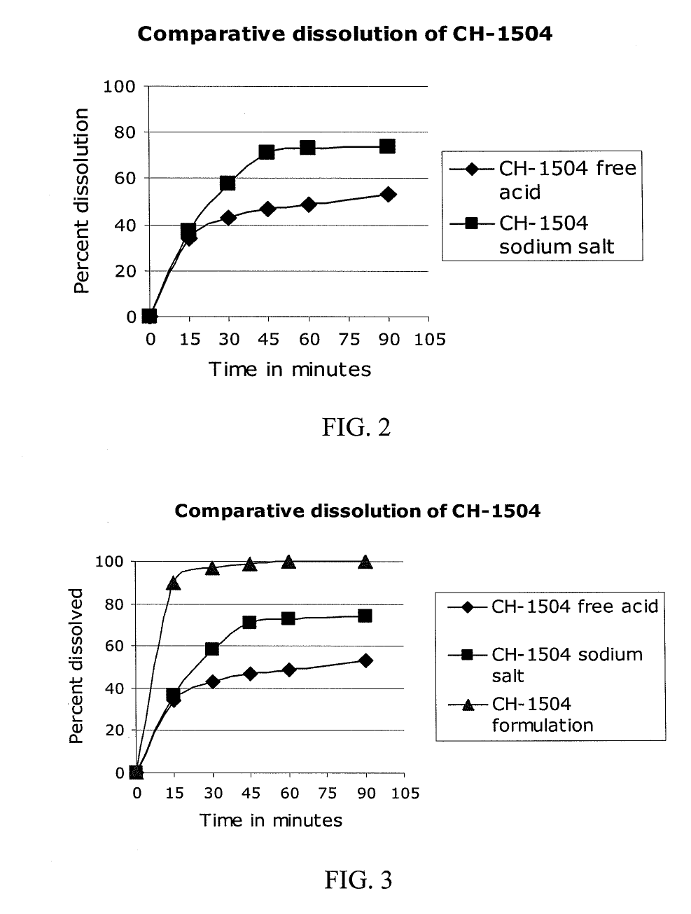

The increased solubility of the sodium salt of the antifolate compounds useful in the invention, such as the disodium salt of the compound of Formula (11), is further illustrated in FIG. 2. Therein is shown the comparative dissolution of the compound of Formula (9), denoted “CH-1504 free acid” and the disodium salt of the compound of Formula (11), denoted “CH-1504 sodium salt”. The percent dissolution for both compounds in 0.1N hydrochloric acid as a function of time was evaluated using a standard USP dissolution apparatus and high performance liquid chromatography (HPLC) test equipment. After a time of about 15 minutes, the sodium salt compound clearly exhibits much greater solubility. By a time of 45 minutes, the sodium salt compound exhibits a percent dissolution of about 70% compared to only 45% for the acid compound. This is particularly relevant in the case of pharmaceutical compositions of the invention for oral delivery, wherein the composition will encounter an acidic environment, such as in the stomach. Greater solubility of the sodium salt compound indicates a greater amount of the active compound will be available for absorption.

Although the invention clearly encompasses compositions comprising compounds in the salt form that are provided in a racemic mixture, in certain embodiments of the invention, it is particularly useful to provide pharmaceutical compositions comprising an antifolate compound that is in the salt form and that is enantiomerically purified for the (S) enantiomer. For example, in one embodiment, the invention provides a pharmaceutical composition comprising a disodium salt or a dipotassium salt of 2-{4-[2-(2,4-diamino-quinazolin-6-yl)-ethyl]-benzoylamino}-4-methylene-pentanedioic acid that is enantiomerically purified for the (S) enantiomer, as described above. Accordingly, in a preferred embodiment, the invention provides a pharmaceutical composition comprising a compound according to Formula (12), which is a salt of (S)-2-{4-[2-(2,4-diamino-quinazolin-6-yl)-ethyl]-benzoylamino}-4-methylene-pentanedioic acid, and wherein X⁺ is as defined above in relation to Formula (11). Preferably, the composition is at least 95% pure for the (S) enantiomer, more preferably at least 97% pure, still more preferably at least 98% pure, even more preferably at least 99% pure, and most preferably at least 99.5% pure for the (S) enantiomer.

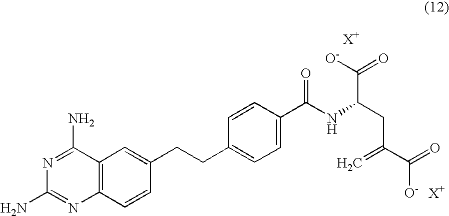

In the case of solid compositions, it is understood that the compounds used in the pharmaceutical compositions of the invention may exist in different forms. For example, the compounds may exist in stable and metastable crystalline forms and isotropic and amorphous forms, all of which are intended to be within the scope of the present invention.

Crystalline and amorphous forms of the inventive compounds can be characterized by the unique X-ray powder diffraction pattern (i.e., interplanar spacing peaks expressed in Angstroms) of the material. Equipment useful for measuring such data is known in the art, such as a Shimadzu XRD-6000 X-ray diffractometer, and any such equipment can be used to measure the compounds according to the present invention.

In specific embodiments, the invention comprises pharmaceutical compositions comprising antifolate compounds, as described above, in a stable crystalline form. In a specific embodiment, the pharmaceutical compositions comprise a salt compound according to Formula (11) in a stable crystalline form. In a preferred embodiment, the pharmaceutical compositions comprise a salt compound according to Formula (12) in a stable crystalline form, and wherein the compound has an enantiomeric purity for the (S) enantiomer as described herein.

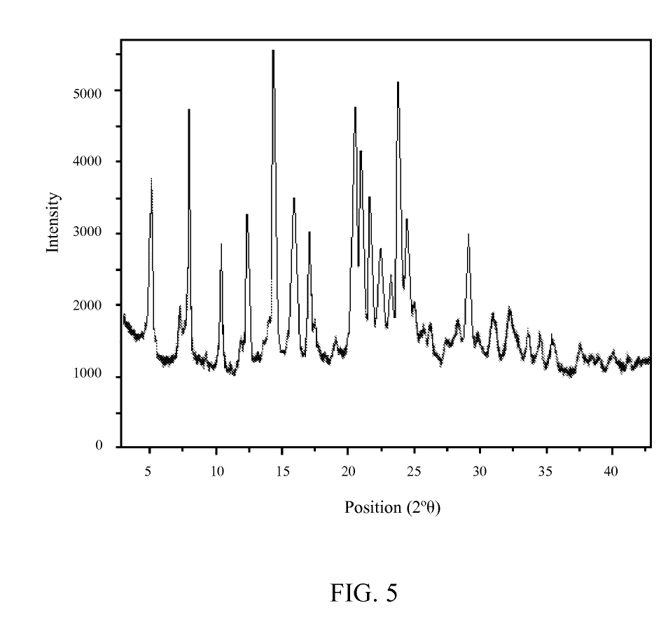

In one embodiment of the invention, an antifolate compound used in the inventive compositions is a disodium salt characterized by the following approximate X-ray powder diffraction “d-spacing” peaks (i.e., interplanar spacing peaks at 2°θ): 4.8750, 7.3490, 8.1221, 10.5019, 11.8701, 12.4449, 14.5270, 16.0326, 17.1551, 20.6738, 21.1909, 21.7468, 22.5306, 23.2841, 23.9665, 24.4918, 28.3375, 29.1428, 30.8958, 32.2118, 33.5960, 34.5226, and 35.4153. The X-ray powder diffraction pattern for this form of the disodium salt is illustrated in FIG. 5 (which is more fully discussed below in Example 1).

The pharmaceutical compositions of the present invention further include prodrugs and active metabolites of the antifolate compounds of the invention. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. In preferred embodiments, the compounds of this invention possess anti-proliferative activity against abnormally proliferating cells, or are metabolized to a compound that exhibits such activity.

A number of prodrug ligands are known. In general, alkylation, acylation, or other lipophilic modification of one or more heteroatoms of the compound, such as a free amine or carboxylic acid residue, reduces polarity and allows passage into cells. Examples of substituent groups that can replace one or more hydrogen atoms on the free amine and/or carboxylic acid moiety include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphatidylcholine; phosphocholine; amino acid residues or derivatives; amino acid acyl residues or derivatives; peptides; cholesterols; or other pharmaceutically acceptable leaving groups which, when administered in vivo, provide the free amine and/or carboxylic acid moiety. Any of these can be used in combination with the disclosed compounds to achieve a desired effect.

Various processes for synthesizing antifolate compounds are disclosed in U.S. Pat. No. 4,996,207, U.S. Pat. No. 5,550,128, Abraham et al. (1991) J. Med. Chem. 34:222-227, and Rosowsky et al. (1991) J. Med. Chem. 34:203-208, all of which are incorporated herein by reference. As one example of a method of synthesis, the compound according to Formula (12) can be prepared according to Reaction Scheme I, shown below.

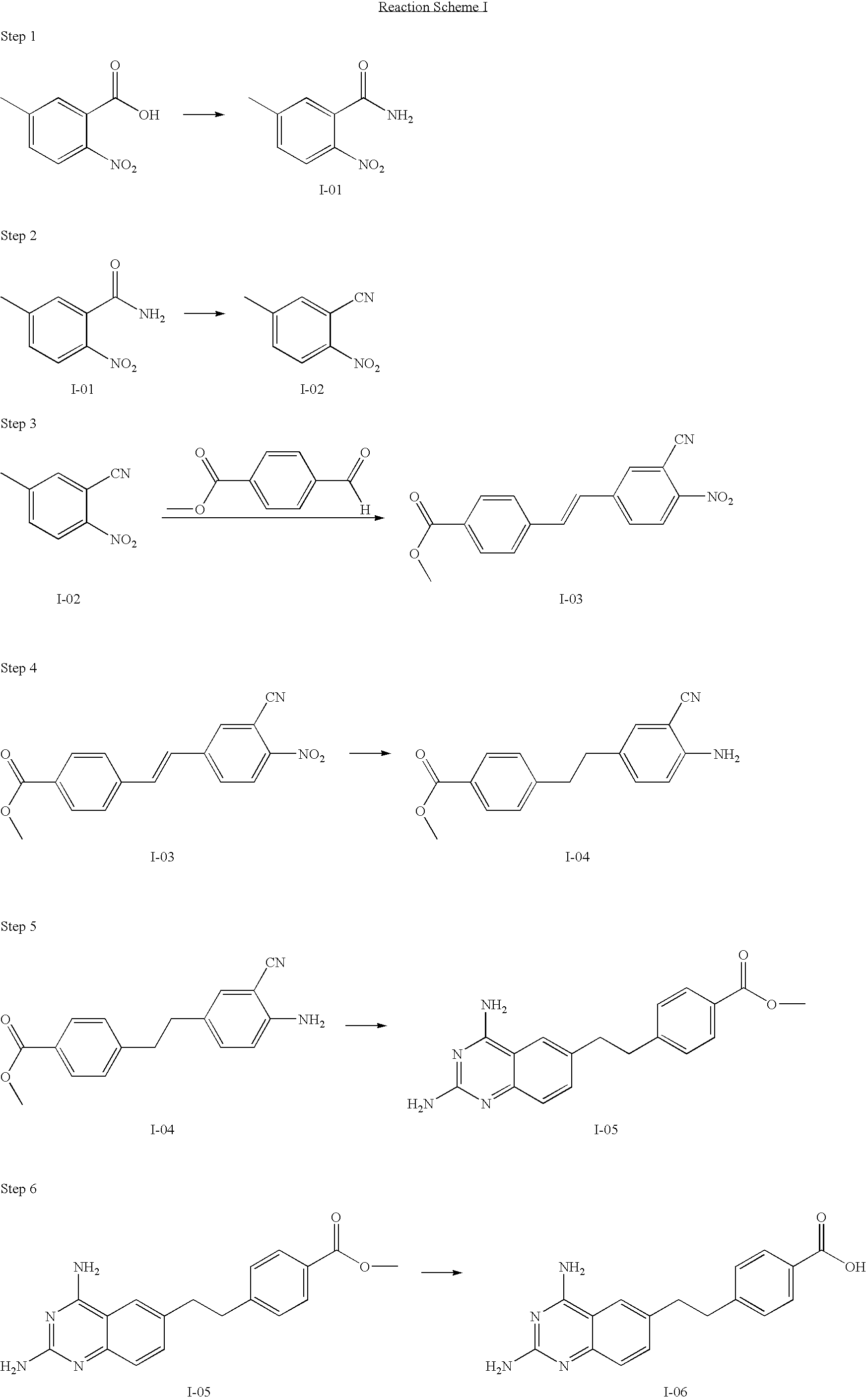

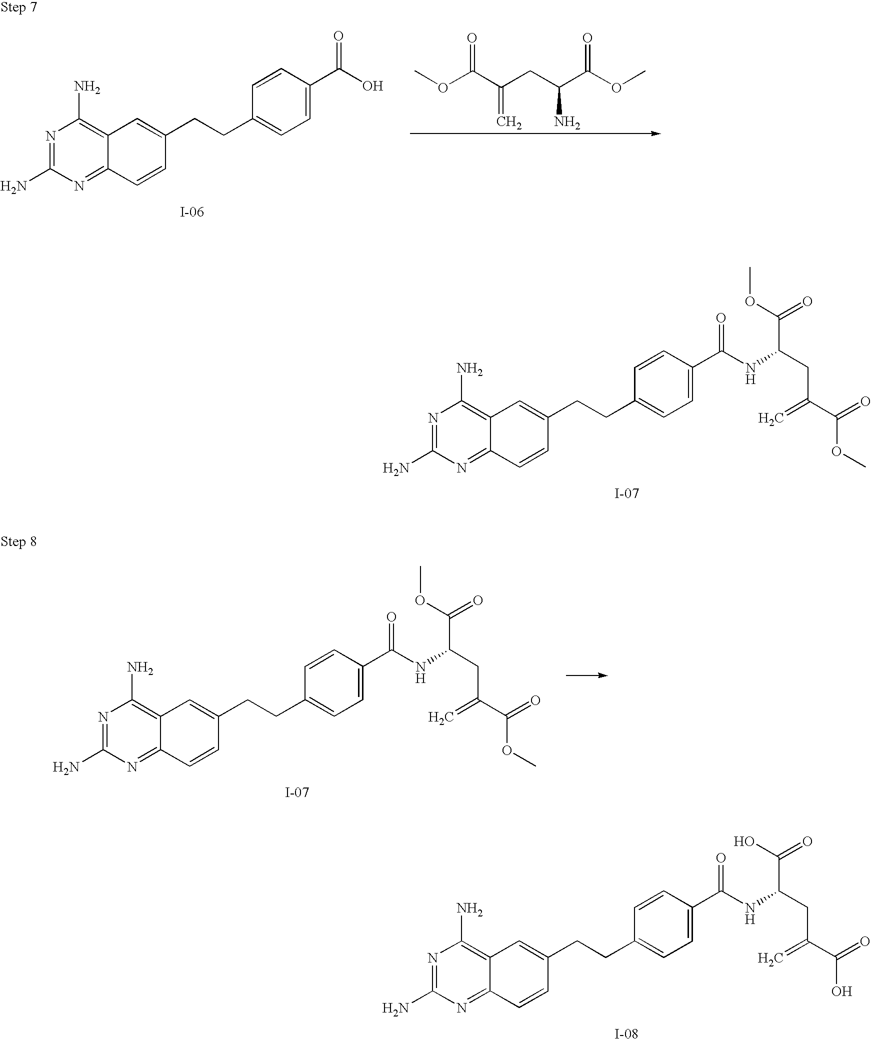

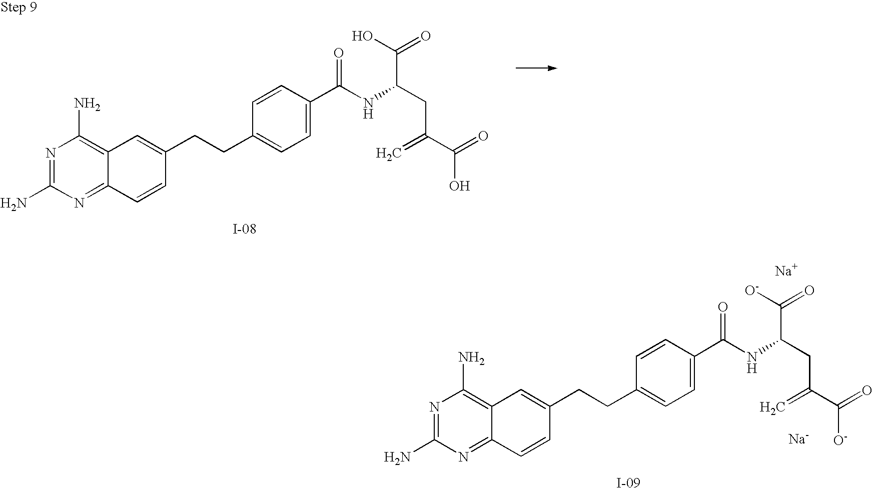

According to Reaction Scheme 1,6-nitro-m-toluic acid is converted to intermediate compound I-01 via reaction with a carboxylate activator, such as isobutyl chloroformate, and triethylamine. Compound I-01 is then converted to the cyanate form (1-02), such as by reacting with phosphorus oxychloride in dimethylformamide. In step 3, compound I-02 is reacted with 4-methoxycarbonyl-benzaldehyde in a suitable solvent, such as tetrahydrofuran, in the presence of a nucleophilic organocatalyst, such as 1,1,3,3-tetramethylguanidine to form compound I-03. This compound is then hydrogenated in the presence of a suitable catalyst, such as carbon-supported palladium, preferably in a suitable solvent, such as tetrahydrofuran, to form compound I-04. In step 5, the fused ring compound I-05 is formed by reacting compound I-04 (in a solution of sulfolane) with chloroformamidine hydrochloride. Compound I-05 is converted to the carboxylic acid compound I-06 (4-[2-(-2,4-diamino-quinazolin-6-yl)ethyl]benzoic acid), such as by refluxing in a base and organic solvent, evaporating the solvent, and acidifying the remaining material. In step 7, compound I-06 is reacted with (S)-2-amino-4-methylene-pentanedioc acid dimethyl ester hydrochloride, 1-(3-dimethylaminopropyl)-3-ethyl carbodiimide hydrochloride, 1-hydroxybenzotriazole, and 4-dimethylaminopyridine in a suitable solvent, such as dimethylformamide, in the presence of a hindered base, such as N,N′-diisopropylethylamine. This reaction results in formation of compound I-07 in the desired enantiomeric form (i.e., the (S) enantiomer). Preferably, the remaining reaction steps are carried out in a manner to preserve this stereochemistry. In step 8, (S)-2-{4-[2-(2,4-diamino-quinazolin-6-yl)-ethyl]benzoylamino}-4-methylene-pentanedioic acid dimethyl ester (compound I-07) is reacted with a base in a suitable solvent, such as acetonitrile to form the corresponding dioic acid of compound I-08. In step 9, the salt compound I-09 is formed by forming a solution using an appropriate solvent, such as methanol, and adding an appropriate base providing the desired cation, such as sodium hydroxide. The salt compound can then be precipitated by conventional means. In one embodiment, the foregoing method can be used to prepare a compound according to Formula (12) as a disodium salt or dipotassium salt having an enantiomeric purity of 99.8% for the (S) enantiomer.

### III. Pharmaceutical Compositions

The present invention particularly provides pharmaceutical compositions comprising one or more antifolate compounds as described herein or pharmaceutically acceptable esters, amides, salts, solvates, analogs, derivatives, or prodrugs thereof. Further, the inventive compositions can be prepared and delivered in a variety of combinations. For example, the composition can comprise a single composition containing all of the active ingredients. Alternately, the composition can comprise multiple compositions comprising separate active ingredients but intended to be administered simultaneously, in succession, or in otherwise close proximity of time.

The pharmaceutical compositions can be prepared to deliver one or more antifolate compounds together with one or more pharmaceutically acceptable carriers therefore, and optionally, other therapeutic ingredients. Carriers should be acceptable in that they are compatible with any other ingredients of the composition and not harmful to the recipient thereof. A carrier may also reduce any undesirable side effects of the agent. Non-limiting examples of carriers that could be used according to the invention are described by Wang et al. (1980) J. Parent. Drug Assn. 34(6):452-462, herein incorporated by reference in its entirety.

In certain embodiments, the pharmaceutical compositions of the invention comprise one or more antifolate compounds, as described herein, in combination with one or more additives useful to increase solubility of the antifolate compound(s) and/or to enhance the bioavailability of the antifolate compound(s). In certain embodiments, the pharmaceutical compositions of the invention comprise one or more antifolate compounds as described herein in combination with a surface active excipient, preferentially a GELUCIRE® compound. In other embodiments, the pharmaceutical compositions of the invention comprise one or more antifolate compounds as described herein in combination with a complexing agent, preferentially a cyclodextrin compound. In still further embodiments, other solubility/bioavailability enhancers could be used. Non-limiting examples of further solubility/bioavailability enhancers include tocopherol (i.e., vitamin-E), polyethyleneglycol compounds (e.g., PEG-4000), polyethylene glycol esters (e.g., LABRAFIL© 1944CS), polyvinylpyrrolidones (e.g., Povidone K29/32), polyethyleneoxide copolymers (e.g., LUTROL© F68), alkyl-pyrrolidones (e.g., PHARMASOLVE® and PHARMASOLVE®-Polysorbate 80), polyoxyethylene esters of fatty acids, such as polyoxyl esters of castor oil (e.g., CREMOPHOR© EL), sorbated vegetable oils (e.g., olive oil—Polysorbate 80), salts and esters of caprylic acid (e.g., CAPTEX® 355-Polysorbate 80 and ACCONON© MC8-2), and microcrystalline cellulose (e.g., AVICEL© PH 101).

GELUCIRE®, a product of Gattefosse s.a., Saint-Priest Cedex, France and Westwood, N.J., USA, is an excipient that is useful in various applications and is available in multiple forms having a range of properties. It is a semi-solid excipient formed of fatty acid esters of glycerol and polyethylene glycol esters (“PEG esters”) and can be described as a polyglycolized glyceride. Accordingly, these terms are also meant to be interchangeable as used herein and are meant to encompass GELUCIRE® compositions. Polyglycolized glycerides are inert semi-solid waxy materials which are amphiphilic in character and are available with varying physical characteristics. They are surface active in nature and disperse or solubilize in aqueous media forming micelles, microscopic globules, or vesicles. They are identified by their melting point/HLB value. The melting point is expressed in degrees Celsius and the HLB (Hydrophile-Lipophile Balance) is a numerical scale extending from 1 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of polyglycolized glyceride excipient may be chosen to achieve the desired characteristics of melting point and/or HLB value. The appropriate choice of melting point/HLB value of a polyglycolized glyceride or a mixture of polyglycolized glyceride compositions will provide the delivery characteristics needed for a specific function, e.g., immediate release, sustained release, and the like.

In certain embodiments, it is preferable to use a polyglycolized glyceride compound having specific characteristics. For example, in specific embodiments, it is useful to choose a particular polyglycolized glyceride compound having a melting point that is less than about 50° C. In other embodiments, the polyglycolized glyceride can have a melting point in the range of about 33° C. to about 50° C. In further embodiments, the polyglycolized glyceride compound can be chosen based upon its HLB value. In specific embodiments, the polyglycolized glyceride compound has an HLB value that is greater than about 8. In other embodiments, the polyglycolized glyceride compound has an HLB value of about 8 to about 14. In even further embodiments, the polyglycolized glyceride can be chosen based upon the type of fatty acid or the type of PEG compound used. For example, it is useful for the fatty acid to be a glyceryl ester, such as glyceryl laurate, although any C₁₄-C₂₀ fatty acid ester could be used. In other embodiments, the PEG compound can be chosen based upon the molecular weight of the PEG compound (which is based on the total number of ethylene glycol groups present in the polymer). For example, the PEG compound can have a number average MW of about 1,200 to about 2,500 Da (i.e., PEG 1,000 to about PEG 2,000). In other embodiments, the PEG compound can range from about PEG 1200 to about PEG 1800, from about PEG 1300 to about PEG 1800, or from about PEG 1400 to about PEG 1600. GELUCIRE® 44/14 is particularly useful according to certain embodiments of the invention and is PEG1500 ester of glyceryl laurate having a melting point of 44° C. and an HLB of 14.

The low melting points of many of the solid polyglycolized glyceride compositions provide a means of incorporating the pharmaceutically active ingredients in them at temperatures from about 0° C. to about 50° C. above their respective melting points. The melt can be filled, for example, in hard gelatin capsules to make the final delivery form. The melt solidifies inside the capsules upon cooling to room temperature. In one embodiment, a pharmaceutical composition of the invention can be prepared by melting the polyglycolized glyceride component and combining the antifolate compound to be included. Any remaining components of the composition can be added while the polyglycolized glyceride is still in the molten state. A pharmaceutical formulation and its method of preparation, according to one embodiment of the invention, incorporating a polyglycolized glyceride is described in Example 9.

In particular embodiments, it can be useful to prepare the formulations using a specific technique dividing certain components of the formulation into an “intragranular” portion and an “extragranular” portion. For example, a portion of the bulking agent and the lubricant (the intragranular portion) can be added to the molten polyglycolized glyceride mixture including the pharmaceutically active antifolate compound. In this mixture, the amount of the bulking agent and the amount of the lubricant can be referred to as a “first amount” of each component. This mixture can be granulated, and the remaining portion of the bulking agent and the lubricant (the extragranular portion or a “second amount” of each component) can then be added to the granulated mixture to form the final composition. The second amount of the bulking agent and the lubricant can be the same or different from the first amount of each component (i.e., the first and second amounts of bulking agent can be the same bulking agent or can be different bulking agents, and the first and second amounts of lubricant can be the same lubricant or can be different lubricants).

Separating certain components into intragranular and extragranular portions for additions at separate stages of the manufacturing process can be particularly beneficial in preparing an end product having desired properties. For example, including a portion of the bulking agent in the extragranular phase is useful for adding bulk to the finished composition. However, adding a porting of the bulking agent in the intragranular phase also has the advantage of increasing drug dispersion within the molten phase. Thus, it is possible to enhance the overall composition.

The amount of polyglycolized glyceride compound used in the pharmaceutical compositions of the invention can vary. In certain embodiments, the amount of polyglycolized glyceride compound is related to the amount of the antifolate compound used. For example, the ratio of polyglycolized glyceride to antifolate compound can be in the range of about 0.1:1 to about 80:1. In specific embodiments, the ratio of polyglycolized glyceride compound to antifolate compound is in the range of about 1:1 to about 50:1, about 2:1 to about 40:1, about 5:1 to about 25:1, or about 10:1 to about 20:1.

In other embodiments, the amount of polyglycolized glyceride compound used in the pharmaceutical formulations of the invention is based on the overall weight of the composition. For example, in certain embodiments, the pharmaceutical compositions of the invention comprise polyglycolized glyceride compound in an amount of up to about 250 mg per gram of overall composition. In further embodiments, the inventive pharmaceutical compositions comprise about 1 mg/g to about 250 mg/g, about 5 mg/g to about 200 mg/g, about 25 mg/g to about 175 mg/g, or about 50 mg/g to about 150 mg/g of polyglycolized glyceride compound, based on the weight of the overall pharmaceutical composition.

Cyclodextrins (originally called cellulosine and now sometimes called cycloamyloses) make up a family of cyclic oligosaccharides composed of 5 or more α-D-glucopyranoside units linked by α-(1,4) glycosidic linkages, as in amylose (a fragment of starch). The smallest (and non-naturally occurring cyclodextrin) is the 5-membered macrocycle. The largest, well-characterized cyclodextrin contains 32 1,4-anhydroglucopyranoside units, but at least 150-membered cyclic oligosaccharides are also known (although generally as a poorly characterized mixture). The most commonly known cyclodextrins contain a number of glucose monomers ranging from six to eight units in a ring. The three naturally occurring cyclodextrins are six, seven, and eight sugar ring molecules typically known as α-cyclodextrin, β-cyclodextrin, and γ-cyclodextrin, respectively. For representative purposes, the chemical structure for β-cyclodextrin is provided below in Formula (13).

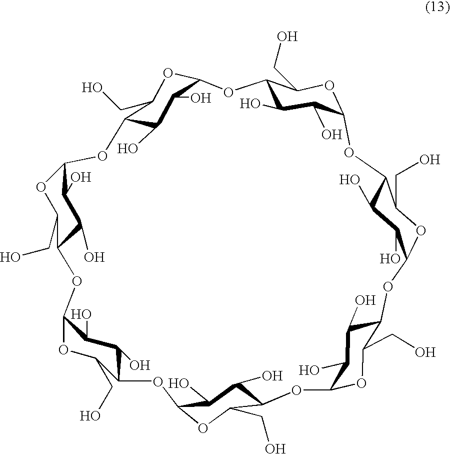

The most stable three dimensional molecular configuration for cyclodextrins in a solvent takes the form of a toroid with the upper (larger) and lower (smaller) opening of the toroid presenting secondary and primary hydroxyl groups, respectively, to the solvent environment. The interior of the toroid is hydrophobic as a result of the electron rich environment provided in large part by the glycosidic oxygen atoms. Cyclodextrins can form stable, aqueous complexes with many compounds, and it is the interplay of atomic (Van der Waals), thermodynamic (hydrogen bonding), and solvent (hydrophobic) forces that is typically believed to account for the stable complexes that may be formed with chemical substances while in the apolar environment of the cyclodextrin cavity. It is this complexing function that makes cyclodextrins particularly useful according to the present invention to enhance solubility and bioavailability of the antifolate compounds. To this end, cyclodextrins can facilitate the formation of a drug-protective micro-environment, create and maintain stable homogeneous distributions, provide more convenient physical forms (e.g., suspension to solution or oil to solid), and alter drug physical properties (e.g., smell and taste). Cyclodextrins are further generally described in Comprehensive Supramolecular Chemistry, Volume 3, Cyclodextrins (Lehn, Jean-Marie and Osa, Tetsuo, editors), Elsevier Science, Inc., which is incorporated herein by reference in its entirety.

Any cyclodextrin compound generally functioning as described above may be used in the compositions of the present invention. In particular, cyclodextrins comprising six to twelve glucose units can be used in the invention. In preferred embodiments, cyclodextrins used in the inventive compositions comprise β-cyclodextrin (BCD), or salts or derivatives thereof. In further embodiment, the cyclodextrins used in the invention can comprise α-cyclodextrin (ACD), or salts or derivatives thereof, or γ-cyclodextrin (GCD), or salts or derivatives thereof. Still further, the cyclodextrins used in the invention can comprise various combinations of one or more BCD, ACD, or GCD (or salts or derivatives thereof).

In addition to unsubstituted cyclodextrins, the compositions of the invention can include one or more cyclodextrin derivatives, such as hydroxypropyl BCD. As used herein, a cyclodextrin derivative refers to a cyclodextrin wherein one or more of the hydroxyl groups have been altered through chemical reaction to introduce one or more different chemical moieties into the cyclodextrin molecule. Non-limiting examples of cyclodextrin derivatives useful according to the invention are described in U.S. Pat. No. 4,727,064, U.S. Pat. No. 5,376,645, and U.S. Pat. No. 6,001,343, all of which are incorporated herein by reference in their entirety.

Cyclodextrins are particularly useful for increasing solubility and bioavailability because of the ease of mixing. For example, β-cyclodextrin is commonly available in a powder form that can simply be blended with additional composition component. A pharmaceutical formulation and its method of preparation, according to one embodiment of the invention, incorporating a cyclodextrin are described in Example 10.

The amount of cyclodextrin compound used in the pharmaceutical compositions of the invention can vary. In certain embodiments, the amount of cyclodextrin compound is related to the amount of the antifolate compound used. For example, the ratio of cyclodextrin to antifolate compound can be in the range of about 1:1 to about 80:1. In specific embodiments, the ratio of cyclodextrin compound to antifolate compound is in the range of about 2:1 to about 50:1, about 5:1 to about 40:1, about 10:1 to about 25:1, or about 10:1 to about 20:1.

In other embodiments, the amount of cyclodextrin compound used in the pharmaceutical formulations of the invention is based on the overall weight of the composition. For example, in certain embodiments, the pharmaceutical compositions of the invention comprise cyclodextrin compound in an amount of up to about 250 mg per gram of overall composition. In further embodiments, the inventive pharmaceutical compositions comprise about 1 mg/g to about 250 mg/g, about 5 mg/g to about 200 mg/g, about 25 mg/g to about 175 mg/g, or about 50 mg/g to about 150 mg/g of cyclodextrin compound, based on the weight of the overall pharmaceutical composition.

In addition to the antifolate compound(s) and the compound(s) added to increase solubility/bioavailability, the pharmaceutical compositions of the present invention can also include further ingredients. Examples of such further ingredients are provided in detail below. In certain embodiments, it is particularly useful for a pharmaceutical composition according to the present invention comprises an antifolate compound as described herein, a solubility/bioavailability enhancer (e.g., a polyglycolized glyceride compound or a cyclodextrin), and one or more of a bulking agent, a lubricant, and an anti-adherent.

Bulking agents are useful to increase the overall content of the composition so that the final dosage form is of a suitable bulk (e.g. to be in the form of a standard sized pill or capsule). Non-limiting examples of bulking agents that may be used in the inventive compositions include carbohydrates and cellulosic materials. Further description of bulking agents is provided otherwise herein. In a specific embodiment, a particularly useful bulking agent is mannitol (such as available under the name PEARLITOL® 100 SD). The content of bulking agent included in the inventive composition can vary. In certain embodiments, the bulking agent is present in a range of about 10% to about 95% by weight, about 50% to about 90% by weight, or about 80% to about 90% by weight.

Lubricants useful according to the invention are also described further below. In certain embodiments, it is useful to include stearic acid and esters thereof as a lubricant. One specific lubricant that may be used is magnesium stearate. The content of lubricant included in the inventive composition can vary. In certain embodiments, the lubricant is present in a range of about 0.25% to about 2% by weight, about 0.5% to about 1% by weight, or about 0.75% to about 1% by weight.

It is also beneficial to include one or more anti-adherent compounds to the formulation, particularly in oral dosage forms, as more fully described herein. One example of an anti-adherent useful according to the invention is colloidal silicon dioxide. The content of anti-adherent included in the inventive composition can also vary. In certain embodiments, the anti-adherent is present in a range of about 0.5% to about 5% by weight, about 0.5% to about 3% by weight, or about 0.5% to about 2% by weight.

The combination of a polyglycolized glyceride compound with a disodium antifolate compound according to Formula (11) has been shown to exhibit greatly increased solubility in comparison to the disodium antifolate compound alone and in comparison to the antifolate compound in the diacid form (e.g., the compound of Formula (9)). Such improved solubility is illustrated in FIG. 3, wherein the comparative dissolution of an antifolate compound is given as the percent dissolution as a function of time. The antifolate compound was tested in the diacid form (denoted as “CH-1504 free acid”), in the sodium salt form (denoted as “CH-1504 sodium salt”), and as the sodium salt form in a pharmaceutical composition according to the invention including GELUCIRE® 44/14 (denoted as “CH-1504 formulation”). Dissolution was tested using 0.1N hydrochloric acid. After 15 minutes, the inventive formulation exhibited approximately 80% dissolution, but the salt alone and the diacid alone only exhibited approximately 35% dissolution after this amount of time. The inventive formulation achieved 90% dissolution by 30 minutes and 100% dissolution by 45 minutes. After 90 minutes, the salt alone and the diacid alone achieved only about 75% dissolution and about 50% dissolution, respectively.

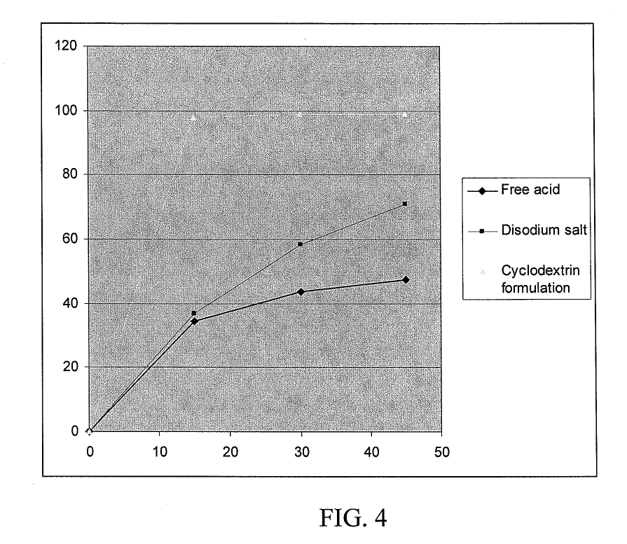

Compositions according to the invention using cyclodextrins have also shown similarly beneficial results. The improved solubility of the inventive compositions comprising an antifolate compound and a cyclodextrin is illustrated in FIG. 4, wherein the comparative dissolution of an antifolate compound is again given as a percent dissolution as a function of time. The antifolate compound was again tested in the diacid form (denoted as “Free acid”), in the sodium salt form (denoted as “Disodium salt”), and as the sodium salt form in a pharmaceutical composition according to the invention including a cyclodextrin (denoted as “Cyclodextrin formulation”). After 15 minutes, the inventive formulation exhibited approximately 95% dissolution, but the salt alone and the diacid alone only exhibited approximately 30-35% dissolution after this amount of time. The inventive formulation approached 100% dissolution within 30 minutes. After 45 minutes, the salt alone and the diacid alone achieved only about 70% dissolution and about 45% dissolution, respectively.

The pharmaceutical compositions of the invention preferably include an antifolate compound in a therapeutically effective amount, as further described below. In certain embodiments, the amount of antifolate compound in the compositions is based on the overall weight of the composition. For example, in certain embodiments, the pharmaceutical composition comprises an antifolate compound in an amount of about 0.01 mg/g to about 100 mg/g. In further embodiments, the pharmaceutical composition comprises an antifolate compound in an amount of about 0.02 mg/g to about 80 mg/g, about 0.05 mg/g to about 75 mg/g, about 0.08 mg/g to about 50 mg/g, about 0.1 mg/g to about 30 mg/g, about 0.25 mg/g to about 25 mg/g, or about 0.5 mg/g to about 20 mg/g. The amount of drug can also be referenced to a unit dose (e.g., the amount of drug in a single capsule or tablet). The content of the antifolate compound can be referenced to the content of the salt. In other embodiments, even when a salt form is used, the amount of the antifolate compound can be referenced to the content of the free acid present.

Compositions of the present invention may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety. Pharmaceutical compositions according to the present invention are suitable for various modes of delivery, including oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrasternal, and transdermal), topical (including dermal, buccal, and sublingual), pulmonary, vaginal, urethral, and rectal administration. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated. In preferred embodiments, the compositions of the present invention are provided in an oral dosage form, as more fully described below.

The pharmaceutical compositions may be conveniently made available in a unit dosage form, whereby such compositions may be prepared by any of the methods generally known in the pharmaceutical arts. Generally speaking, such methods of preparation comprise combining (by various methods) the active compounds of the invention with a suitable carrier or other adjuvant, which may consist of one or more ingredients. The combination of the active ingredients with the one or more adjuvants is then physically treated to present the composition in a suitable form for delivery (e.g., shaping into a tablet or forming an aqueous suspension).

Pharmaceutical compositions according to the present invention suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

In one embodiment, compound may be administered orally in combination with a pharmaceutically acceptable vehicle such as an inert diluent or an edible carrier. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

Hard capsules containing the compound may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present invention may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT®), zein, shellac, and polysaccharides.

Powdered and granular compositions of a pharmaceutical preparation of the invention may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of the pharmaceutical composition of the invention which are suitable for oral administration may be prepared, packaged, and sold either in liquid form or in the form of a dry product intended for reconstitution with water or another suitable vehicle prior to use.

A tablet containing one or more compounds according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Adjuvants or accessory ingredients, in addition to those discussed above, for use in the compositions of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the composition according to the present invention may be anionic, cationic, amphoteric, or nonionic surface active agents. Stabilizers may be included in the compositions to inhibit or lessen reactions leading to decomposition of the active agents, such as oxidative reactions.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, the pharmaceutical compositions of the invention can be administered intermittently. Administration of the therapeutically effective dose may be achieved in a continuous manner, as for example with a sustained-release composition, or it may be achieved according to a desired daily dosage regimen, as for example with one, two, three, or more administrations per day. By “time period of discontinuance” is intended a discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

The inventive pharmaceutical compositions can comprise a single pharmaceutically active antifolate compound as described herein, can comprise two or more pharmaceutically active antifolate compounds as described herein, or can comprise one or more pharmaceutically active antifolate compounds as described herein with one or more further pharmaceutically active compounds (i.e., co-administration). Accordingly, it is recognized that the pharmaceutically active compounds in the compositions of the invention can be administered in a fixed combination (i.e., a single pharmaceutical composition that contains both active materials). Alternatively, the pharmaceutically active compounds may be administered simultaneously (i.e., separate compositions administered at the same time). In another embodiment, the pharmaceutically active compounds are administered sequentially (i.e., administration of one or more pharmaceutically active compounds followed by separate administration or one or more pharmaceutically active compounds). One of skill in the art will recognized that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to treat abnormal cell proliferation. In another embodiment, a therapeutically effective amount is an amount effective to treat inflammation. In yet another embodiment, a therapeutically effective amount is an amount effective to treat arthritis. In still another embodiment, a therapeutically effective amount is an amount effective to treat asthma.

The active compound is included in the pharmaceutical composition in an amount sufficient to deliver to a patient a therapeutic amount of a compound of the invention in vivo in the absence of serious toxic effects. The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time

A therapeutically effective amount according to the invention can be determined based on the bodyweight of the recipient. For example, in one embodiment, a therapeutically effective amount of one or more compounds of the invention is in the range of about 0.1 μg/kg of body weight to about 5 mg/kg of body weight per day. Alternatively, a therapeutically effective amount can be described in terms of a fixed dose. Therefore, in another embodiment, a therapeutically effective amount of one or more compounds of the invention is in the range of about 0.01 mg to about 500 mg per day. Of course, it is understood that such an amount could be divided into a number of smaller dosages administered throughout the day. The effective dosage range of pharmaceutically acceptable salts and prodrugs can be calculated based on the weight of the parent antifolate to be delivered. If a salt or prodrug exhibits activity in itself, the effective dosage can be estimated as above using the weight of the salt or prodrug, or by other means known to those skilled in the art.

It is contemplated that the compositions of the invention comprising one or more compounds described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

### IV. Active Agent Combinations

For use in treating various diseases or conditions, the pharmaceutical compositions of the invention can include the antifolate compounds described above in various combinations. For example, in one embodiment, a pharmaceutical composition according to the invention can comprise a single antifolate compound described herein, such as the compound according to Formula (12). In another embodiment, a pharmaceutical composition according to the invention can comprise two or more antifolate compounds disclosed herein. In still further embodiments, a pharmaceutical composition according to the invention can comprise one or more antifolate compounds described herein with one or more further compounds known to have therapeutic properties. For example, the pharmaceutical compositions described herein can be administered with one or more toxicity-reducing compounds (e.g., folic acid or leucovorin). In further embodiments, the inventive pharmaceutical compositions can be administered with one or more compounds known to be an anti-inflammatory, anti-arthritic, antibiotic, antifungal, or antiviral agent. Such further compounds can be provided as a component of the pharmaceutical composition or can be provided in alternation with the compositions of the invention. In other words, the pharmaceutical compositions of the invention can be administered with the additional active agent(s) in the same composition with the antifolate compounds disclosed herein, or the additional active agent(s) can be administered in a separate delivery form from the pharmaceutical compositions of the invention. In particular embodiments, the pharmaceutical compositions of the invention can be provided in combination with one or more compounds selected from the groups described below.

In the following description, certain compounds useful as further active agents in the pharmaceutical compositions of the invention with the antifolate compounds disclosed above may be described in reference to specific diseases or conditions commonly treated using the noted compounds. The disclosure of such diseases or conditions is not intended to limit the scope of the invention and particularly does not limit the diseases or conditions that may be treated using the pharmaceutical compositions disclosed herein. Rather such exemplary diseases or conditions are provided only to illustrate the types of diseases and conditions typically treated using the additional compounds.

As additional active agents, the pharmaceutical compositions of the present invention can, in certain embodiments, be administered with antiproliferative agents. Proliferative disorders are currently treated by a variety of classes of compounds including alkylating agents, antimetabolites, natural products, enzymes, biological response modifiers, miscellaneous agents, radiopharmaceuticals (for example, Y-90 tagged to hormones or antibodies), hormones and antagonists. Any of the antiproliferative agents listed below or any other such therapeutic agents and principles as described in, for example, DeVita, V. T., Jr., Hellmann, S., Rosenberg, S. A.; Cancer: Principles & Practice of Oncology, 5th ed., Lippincott-Raven Publishers (1997), can be used with the pharmaceutical compositions of the present invention

Representative, nonlimiting examples of anti-angiogenesis agents suitable for use with the pharmaceutical compositions of the present invention include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs (I-azetidine-2-carboxylic acid (LACA), cis-hydroxyproline), d,1-3,4-dehydroproline, thiaproline, alpha,alpha-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chimp-3, chymostatin, beta-cyclodextrin tetradecasulfate, eponemycin, fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide, angiostatic steroid, carboxynaminolmidazole, and metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

Representative, nonlimiting examples of alkylating agents suitable for use with the pharmaceutical compositions of the present invention include, but are not limited to, Nitrogen Mustards, such as Mechlorethamine (Hodgkin's disease, non-Hodgkin's lymphomas), Cyclophosphamide, Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), Melphalan (L-sarcolysin) (multiple myeloma, breast, ovary), Chlorambucil (chronic lymphocytic leukemia, primary macroglobulinemia, Hodgkin's disease, non-Hodgkin's lymphomas), Ethylenimines and Methylmelamines, such as, Hexamethylmelamine (ovary), Thiotepa (bladder, breast, ovary), Alkyl Sulfonates, such as, Busulfan (chronic granulocytic leukemia), Nitrosoureas, such as, Carmustine (BCNU) (Hodgkin's disease, non-Hodgkin's lymphomas, primary brain tumors, multiple myeloma, malignant melanoma), Lomustine (CCNU) (Hodgkin's disease, non-Hodgkin's lymphomas, primary brain tumors, small-cell lung), Semustine (methyl-CCNU) (primary brain tumors, stomach, colon), Streptozocin (STR) (malignant pancreatic insulinoma, malignant carcinoin) and Triazenes, such as, Dacarbazine (DTIC—dimethyltriazenoimidazole-carboxamide) (malignant melanoma, Hodgkin's disease, soft-tissue sarcomas).

Representative, nonlimiting examples of anti-metabolite agents suitable for use with the pharmaceutical compositions of the present invention include, but are not limited to, Folic Acid Analogs, such as, Methotrexate (amethopterin) (acute lymphocytic leukemia, choriocarcinoma, mycosis fungoides, breast, head and neck, lung, osteogenic sarcoma), Pyrimidine Analogs, such as Fluorouracil (5-fluorouracil-5-FU) Floxuridine (fluorodeoxyuridine —FUdR) (breast, colon, stomach, pancreas, ovary, head and neck, urinary bladder, premalignant skin lesions) (topical), Cytarabine (cytosine arabinoside) (acute granulocytic and acute lymphocytic leukemias), Purine Analogs and Related Inhibitors, such as, Mercaptopurine (6-mercaptopurine-6-MP) (acute lymphocytic, acute granulocytic and chronic granulocytic leukemia), Thioguanine (6-thioguanine-TG) (acute granulocytic, acute lymphocytic and chronic granulocytic leukemia), Pentostatin (2′-deoxycoformycin) (hairy cell leukemia, mycosis fungoides, chronic lymphocytic leukemia), Vinca Alkaloids, such as, Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung), Epipodophyllotoxins, such as Etoposide (testis, small-cell lung and other lung, breast, Hodgkin's disease, non-Hodgkin's lymphomas, acute granulocytic leukemia, Kaposi's sarcoma), and Teniposide (testis, small-cell lung and other lung, breast, Hodgkin's disease, non-Hodgkin's lymphomas, acute granulocytic leukemia, Kaposi's sarcoma).

Representative, nonlimiting examples of cytotoxic agents suitable for use with the pharmaceutical compositions of the present invention include, but are not limited to: doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci.

Representative, non-limiting examples of natural products suitable for use with the pharmaceutical compositions of the present invention include, but are not limited to: Antibiotics, such as, Dactinomycin (actinomycin D) (choriocarcinoma, Wilms' tumor rhabdomyosarcoma, testis, Kaposi's sarcoma), Daunorubicin (daunomycin-rubidomycin) (acute granulocytic and acute lymphocytic leukemias), Doxorubicin (soft tissue, osteogenic, and other sarcomas, Hodgkin's disease, non-Hodgkin's lymphomas, acute leukemias, breast, genitourinary thyroid, lung, stomach, neuroblastoma), Bleomycin (testis, head and neck, skin and esophagus lung, and genitourinary tract, Hodgkin's disease, non-Hodgkin's lymphomas), Plicamycin (mithramycin) (testis, malignant hypercalcemia), Mitomycin (mitomycin C) (stomach, cervix, colon, breast, pancreas, bladder, head and neck), Enzymes, such as, L-Asparaginase (acute lymphocytic leukemia), and Biological Response Modifiers, such as, Interferon-alpha (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia).

Additional agents that can be used with the pharmaceutical compositions disclosed herein include, but are not limited to: Platinum Coordination Complexes, such as, Cisplatin (cis-DDP) Carboplatin (testis, ovary, bladder, head and neck, lung, thyroid, cervix, endometrium, neuroblastoma, osteogenic sarcoma); Anthracenedione, such as Mixtozantrone (acute granulocytic leukemia, breast); Substituted Urea, such as, Hydroxyurea (chronic granulocytic leukemia, polycythemia vera, essential thrombocytosis, malignant melanoma); Methylhydrazine Derivatives, such as, Procarbazine (N-methylhydrazine, MIH) (Hodgkin's disease); Adrenocortical Suppressants, such as, Mitotane (o,p′-DDD) (adrenal cortex), Aminoglutethimide (breast); Adrenorticosteriods, such as, Prednisone (acute and chronic lymphocytic leukemias, non-Hodgkin's lymphomas, Hodgkin's disease, breast); Progestins, such as, Hydroxyprogesterone caproate, Medroxyprogesterone acetate, Megestrol acetate (endometrium, breast); and Steroids, such as betamethasone sodium phosphate and betamethasone acetate.

Representative, nonlimiting examples of hormones and antagonists suitable for use with the pharmaceutical compositions of the present invention include, but are not limited to, Estrogens: Diethylstilbestrol Ethinyl estradiol (breast, prostate); Antiestrogen: Tamoxifen (breast); Androgens: Testosterone propionate Fluxomyesterone (breast); Antiandrogen: Flutamide (prostate); Gonadotropin-Releasing Hormone Analog: and Leuprolide (prostate). Other hormones include medroxyprogesterone acetate, estradiol, megestrol acetate, ocreotide acetate, diethylstilbestrol diphosphate, testolactone, and goserelin acetate.

The pharmaceutical compositions of the present invention can be used with therapeutic agents used to treat arthritis. Examples of such agents include, but are not limited to, the following:

Nonsteroidal anti-inflammatory drugs (NSAIDs), such as cyclooxygenase-2 (COX-2) inhibitors, aspirin (acetylsalicylic acid), ibuprofen, ketoprofen, naproxen, and acetaminophen;

Analgesics, such as acetaminophen, opioid analgesics, and transdermal fentanyl;

Biological response modifiers, such as etanercept, infliximab, adalimumab, anakinra, abatacept, tiruximab, certolizumab pegol, and tocilizumab;

Corticosteroids or steroids, such as glucocorticoids (GC), fluticasone, budesonide, prednisolone, hydrocortisone, adrenaline, Aldosterone, Cortisone Acetate, Desoxymethasone, Dexamethasone, Fluocortolone, Hydrocortisone, Meprednisone, Methylprednisolone, Prednisolone, Prednisone, Prednylidene, Procinonide, Rimexolone, and Suprarenal Cortex;

Disease-modifying antirheumatic drugs (DMARDs), such as hydroxychloroquine, cyclosphosphamide, chlorambucil, the gold compound auranofin, sulfasalazine, minocycline, cyclosporine, toll-like receptor agonists and antagonists, kinase inhibitors (e.g., p38 MAPK) immunosuppressants and tumor necrosis factor (TNF) blockers (e.g., etanercept, infliximab, and adalimumab);

Fibromyalgia medications, such as amitriptyline, fluoxetine, cyclobenzaprine, tramadol, gabapentin, pregabalin, and dual-reuptake inhibitors;

Osteoporosis medications, such as estrogens, parathyroid hormones, bisphosphonates, selective receptor molecules, and bone formation agents;

Gout medications, such as allopurinol, probenecid, losartan, and fenofibrate;

Psoriasis medications, such as acitretin; and

Topical treatments, such as topical NSAIDs and capsaicin.

The pharmaceutical compositions of the present invention also can be used with therapeutic agents used to treat asthma. Examples of such agents include, but are not limited to, the following:

Anti-allergics, such as cromolyn sodium and ketotifen fumarate;

Anti-inflammatories, such as NSAIDs and steroidal anti-inflammatories (e.g., beclomethasone dipropionate, budesonide, dexamethasone sodium phosphate, flunisolide, fluticasone propionate, and triamcinolone acetonide);

Anticholinergics, such as ipratropium bromide, belladonna alkaloids, atropine, and oxitropium bromide;

Antihistamines, such as chlorpheniramine, brompheniramine, diphenhydramine, clemastine, dimenhydrinate, cetirizine, hydroxyzine, meclizine, fexofenadine, loratadine, and enadine;

β₂-adrenergic agonists (beta agonists), such as albutamol, terbutaline, epinephrine, metaproterenol, ipratropium bromide, ephedra (source of alkaloids), ephedrine, and pseudoephedrine;

Leukotriene Receptor Antagonists, such as zafirlukast and zileuton montelukast;

Xanthines (bronchodilators), such as theophylline, dyphylline, and oxtriphylline; Miscellaneous anti-asthma agents, such as xanthines, methylxanthines, oxitriphylline, aminophylline, phosphodiesterase inhibitors such as zardaverine, calcium antagonists such as nifedipine, and potassium activators such as cromakalim; and

Prophylactic agent(s), such as sodium cromoglicate, cromolyn sodium, nedocromil, and ketotifen.

Further, non-limiting examples of active agents that can be used with the pharmaceutical compositions of the present invention include anti-psoriasis agents, anti-Inflammatory Bowel Disease (anti-IBD) agents, anti-chronic obstructive pulmonary disease (anti-COPD) agents, anti-multiple sclerosis agents.

### V. Articles of Manufacture

The present invention also includes an article of manufacture providing a pharmaceutical compositions comprising one or more antifolate compounds disclosed herein, optionally in combination with one or more further active agents. The article of manufacture can include a vial or other container that contains a composition suitable for use according to the present invention together with any carrier, either dried or in liquid form. In particular, the article of manufacture can comprise a kit including a container with a composition according to the invention. In such a kit, the composition can be delivered in a variety of combinations. For example, the composition can comprise a single dosage comprising all of the active ingredients. Alternately, where more than one active ingredient is provided, the composition can comprise multiple dosages, each comprising one or more active ingredients, the dosages being intended for administration in combination, in succession, or in other close proximity of time. For example, the dosages could be solid forms (e.g., tablets, caplets, capsules, or the like) or liquid forms (e.g., vials), each comprising a single active ingredient, but being provided in blister packs, bags, or the like, for administration in combination.

The article of manufacture further includes instructions in the form of a label on the container and/or in the form of an insert included in a box in which the container is packaged, for the carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

### VI. Methods of Treatment

As previously noted, antifolates can vary as to the folate-dependant metabolic process inhibited thereby, and many antifolates act on a variety of enzymes. Pemetrexed (also known as ALIMTA® or L-glutamic acid, N-[4-[2-(2-amino-4,7-dihydro-4-oxo-1H-pyrrolo[2,3-d]pyrimidin-5-yl)ethyl]benzoyl-, disodium salt, heptahydrate) is one example of an antifolate known to act on multiple enzymes. In particular, pemetrexed is known to exhibit antineoplastic activity by inhibiting TS, DHFR, and GARFT.

Thymidylate synthase (TS) is a rate-limiting enzyme in pyrimidine de novo deoxynucleotide biosynthesis and is therefore often a target for chemotherapeutic strategies. In DNA synthesis, TS plays a central role in reductive methylation of deoxyuridine-5′-monophosphate (dUMP) to deoxythymidine-5′-monophosphate (dTMP). Thus, TS inhibition leads directly to depletion of dTMP and subsequently of 2′-deoxythymidine-5′-triphosphate (dTTP), an essential precursor for DNA. This indirectly results in an accumulation of 2′-deoxyuridine-5′-triphosphate (dUTP) and, therefore, leads to so-called “thymine-less death” due to misincorporation of dUTP into DNA and subsequent excision catalyzed by uracil-DNA glycosylase, which causes DNA damage. Both this DNA damage and the noted imbalance in dTTP/dUTP can induce downstream events, leading to apoptosis (cell death).

Dihydrofolate reductase (DHFR) catalyzes the NADPH-dependent reduction of 7,8-dihydrofolate (DHF or H₂F) to 5,6,7,8-tetrahydrofolate (THF or H₄F). Thus, DHFR is necessary for maintaining intracellular levels of THF, an essential cofactor in the synthetic pathway of purines, thymidylate, and several amino acids.

Glycinamide ribonucleotide formyltranferase (GARFT) is a folate-dependent enzyme in the de novo purine biosynthesis pathway critical to cell division and proliferation. Specifically, GARFT catalyzes the formation of purines from the reaction of 10-formyltetrahydrofolate (10-FTHF) to THF. Inhibition of GARFT results in a depletion in intracellular purine levels, which in turn inhibits DNA and RNA synthesis. Ultimately, disruption of DNA and RNA synthesis by GARFT inhibition results in cell death. The antiproliferative effect associated with GARFT inhibition makes it a particularly desirable target for anti-tumor drugs.

Antifolates, such as pemetrexed, can be transported into cells by mechanisms such as the reduced folate carrier system and the membrane folate binding protein transport system. Once in the cell, pemetrexed is converted to polyglutamylate forms by folyl polyglutamate synthase. The polyglutamylate forms are retained in cells and are inhibitors of TS and GARFT. Polyglutamylation is a time- and concentration-dependent process that occurs in tumor cells and, to a lesser extent, in normal tissues. Polyglutamylated metabolites have an increased intracellular half-life resulting in prolonged drug action in malignant cells.

In many instances, broad action against multiple enzymes may not be desirable. For example, pemetrexed inhibits DHFR, TS, and GARFT. As described above, inhibition of TS and GARFT is strongly related to cell death, thus the desirability of using TS and GARFT inhibitors as anti-tumor drugs. However, the ability of drugs, such as pemetrexed, to induce apoptosis increases the toxicity of the drug (i.e., death of healthy cells as well as tumor cells).

The function of compounds, such as pemetrexed, as inhibitors of TS and GARFT arises from the polyglutamylation of the compound inside the cell. Accordingly, compounds that are non-polyglutamylatable would not be expected to function as a TS inhibitor or a GARFT inhibitor. However, inhibition of polyglutamylation does not generally affect the ability of a compound to function as a DHFR inhibitor. For example, pemetrexed has been shown to have equivalent DHFR inhibition in comparison to the polyglutamate forms of pemetrexed.

The antifolate compounds used in the pharmaceutical compositions of the invention comprise a 4-methylidene group in the glutamate moiety of the compounds. Such may also be referred to as a gamma methylene glutamate moiety. The presence of the methylene group makes the antifolate compounds non-polyglutamylatable. Accordingly, the compounds of the invention are specific for DHFR inhibition (i.e., do not inhibit TS or GARFT due to the absence of polyglutamylation inside cells). Such specificity is desirable to provide for more specific treatments while avoiding or reducing toxicity and minimizing side-effects more commonly associated with compounds, such as pemetrexed, which act on additional enzymes, such as TS and GARFT.

The antifolate compounds used in the pharmaceutical compositions of the present invention are particularly useful in the treatment of various conditions wherein disruption of folic acid metabolism is beneficial for treating a symptom of the condition or the condition generally. Accordingly, in further embodiments, the present invention is directed to methods of treating various diseases or conditions. In particular embodiments, the invention provides methods of treating diseases or conditions known or found to be treatable by disruption of folic acid metabolism. In specific embodiments, the invention provides methods of treating conditions, such as abnormal cell proliferation, inflammation (including inflammatory bowel disease), arthritis (particularly rheumatoid arthritis), psoriasis, and asthma.

A. Abnormal Cellular Proliferation

Abnormal cell proliferation has been shown to be the root of many diseases and conditions, including cancer and non-cancer disorders which present a serious health threat. Generally, the growth of the abnormal cells, such as in a tumor, exceeds and is uncoordinated with that of normal cells. Furthermore, the abnormal growth of tumor cells generally persists in an abnormal (i.e., excessive) manner after the cessation of stimuli that originally caused the abnormality in the growth of the cells. A benign tumor is characterized by cells that retain their differentiated features and do not divide in a completely uncontrolled manner. A benign tumor is usually localized and nonmetastatic. A malignant tumor (i.e., cancer) is characterized by cells that are undifferentiated, do not respond to the body's growth control signals, and multiply in an uncontrolled manner. Malignant tumors are invasive and capable of metastasis.

Treatment of diseases or conditions of abnormal cellular proliferation comprises methods of killing, inhibiting, or slowing the growth or increase in size of a body or population of abnormally proliferative cells (including tumors or cancerous growths), reducing the number of cells in the population of abnormally proliferative cells, or preventing the spread of abnormally proliferative cells to other anatomic sites, as well as reducing the size of a growth of abnormally proliferative cells. The term “treatment” does not necessarily mean to imply a cure or a complete abolition of the disorder of abnormal cell proliferation. Prevention of abnormal cellular proliferation comprises methods which slow, delay, control, or decrease the likelihood of the incidence or onset of disorders of abnormal cell proliferation, in comparison to that which would occur in the absence of treatment.

Abnormal cellular proliferation, notably hyperproliferation, can occur as a result of a wide variety of factors, including genetic mutation, infection, exposure to toxins, autoimmune disorders, and benign or malignant tumor induction. Hyperproliferative cell disorders include, but are not limited to, skin disorders, blood vessel disorders, cardiovascular disorders, fibrotic disorders, mesangial disorders, autoimmune disorders, graft-versus-host rejection, tumors, and cancers.

Representative, non-limiting types of non-neoplastic abnormal cellular proliferation disorders that can be treated using the present invention include: skin disorders such as psoriasis, eczema, keratosis, basal cell carcinoma, and squamous cell carcinoma; disorders of the cardiovascular system such as hypertension and vasculo-occlusive diseases (e.g., atherosclerosis, thrombosis and restenosis); blood vessel proliferative disorders such as vasculogenic (formation) and angiogenic (spreading) disorders which result in abnormal proliferation of blood vessels, such as antiogenesis; and disorders associated with the endocrine system such as insulin resistant states including obesity and diabetes mellitus (types 1 & 2).

The compositions and methods of the present invention are also useful for treating inflammatory diseases associated with non-neoplastic abnormal cell proliferation. These include, but are not limited to, inflammatory bowel disease (IBD), rheumatoid arthritis (RA), multiple sclerosis (MS), proliferative glomerulonephritis, lupus erythematosus, scleroderma, temporal arteritis, thromboangiitis obliterans, mucocutaneous lymph node syndrome, asthma, host versus graft, thyroiditis, Grave's disease, antigen-induced airway hyperactivity, pulmonary eosinophilia, Guillain-Barre syndrome, allergic rhinitis, myasthenia gravis, human T-lymphotrophic virus type 1-associated myelopathy, herpes simplex encephalitis, inflammatory myopathies, atherosclerosis, and Goodpasture's syndrome.

In a particular embodiment, the pharmaceutical compositions of the present invention are useful in the treatment of psoriasis. Psoriasis is an immune-mediated skin disorder characterized by chronic T-cell stimulation by antigen-presenting cells (APC) occurs in the skin. The various types of psoriasis include, for example, plaque psoriasis (i.e., vulgaris psoriasis), pustular psoriasis, guttate psoriasis, inverse psoriasis, erythrodermic psoriasis, psoriatic arthritis, scalp psoriasis and nail psoriasis. Common systemic treatments for psoriasis include methotrexate, cyclosporin and oral retinoids, but their use is limited by toxicity. Up to 40% of patients with psoriasis also develop psoriatic arthritis (Kormeili T et al. Br J Dermatol. (2004) 151(1):3-15.

In further embodiments, the pharmaceutical compositions of the present invention are useful in the treatment of blood vessel proliferative disorders, including vasculogenic (formation) and angiogenic (spreading) disorders which result in abnormal proliferation of blood vessels. Other blood vessel proliferative disorders include arthritis and ocular diseases such as diabetic retinopathy. Abnormal neovascularization is also associated with solid tumors. In a particular embodiment, the compositions of the present invention are useful in the treatment of diseases associated with uncontrolled angiogenesis. Representative, non-limiting diseases of abnormal angiogenesis include rheumatoid arthritis, ischemic-reperfusion related brain edema and injury, cortical ischemia, ovarian hyperplasia and hypervascularity, (polycystic ovary syndrome), endometriosis, psoriasis, diabetic retinopathy, and other ocular angiogenic diseases such as retinopathy of prematurity (retrolental fibroplastic), macular degeneration, corneal graft rejection, neuromuscular glaucoma, and Oster Webber syndrome. Cancers associated with abnormal blood cell proliferation include hemangioendotheliomas, hemangiomas, and Kaposi's sarcoma.

In further embodiments, the pharmaceutical compositions of the present invention are useful in the treatment of disorders of the cardiovascular system involving abnormal cell proliferation. Such disorders include, for example, hypertension, vasculo-occlusive diseases (e.g., atherosclerosis, thrombosis, and restenosis after angioplasty), acute coronary syndromes (such as unstable angina, myocardial infarction, ischemic and non-ischemic cardiomyopathies, post-MI cardiomyopathy, and myocardial fibrosis), and substance-induced cardiomyopathy.

Vascular injury can also result in endothelial and vascular smooth muscle cell proliferation. The injury can be caused by traumatic events or interventions (e.g., angioplasty, vascular graft, anastomosis, organ transplant) (Clowes A et al. A. J. Vasc. Surg (1991) 13:885). Restenosis (e.g., coronary, carotid, and cerebral lesions) is the main complication of successful balloon angioplasty of the coronary arteries. It is believed to be caused by the release of growth factors as a result of mechanical injury to the endothelial cells lining the coronary arteries.

Other atherosclerotic conditions which can be treated or prevented by means of the present invention include diseases of the arterial walls that involve proliferation of endothelial and/or vascular smooth muscle cells, including complications of diabetes, diabetic glomerulosclerosis, and diabetic retinopathy.

In further embodiments, the pharmaceutical compositions of the present invention are useful in the treatment of abnormal cell proliferation disorders associated the endocrine system. Such disorders include, for example, insulin resistant states including obesity, diabetes mellitus (types 1 & 2), diabetic retinopathy, macular degeneration associated with diabetes, gestational diabetes, impaired glucose tolerance, polycystic ovarian syndrome, osteoporosis, osteopenia, and accelerated aging of tissues and organs including Werner's syndrome.

In further embodiments, the pharmaceutical compositions of the present invention are useful in the treatment of abnormal cell proliferation disorders of the urogenital system. These include, for example, endometriosis, benign prostatic hyperplasia, eiomyoma, polycystic kidney disease, and diabetic nephropathy.

In further embodiments, the pharmaceutical compositions of the present invention are useful in the treatment of fibrotic disorders. Medical conditions involving fibrosis include undesirable tissue adhesion resulting from surgery or injury. Non-limiting examples of fibrotic disorders include hepatic cirrhosis and mesangial proliferative cell disorders.

In still further embodiments, abnormal cell proliferation disorders of the tissues and joints can be treated according to the present invention. Such disorders include, for example, Raynaud's phenomenon/disease, Sjogren's Syndrome systemic sclerosis, systemic lupus erythematosus, vasculitides, ankylosing spondylitis, osteoarthritis, reactive arthritis, psoriatic arthritis, and fibromyalgia.

In certain embodiments, abnormal cell proliferation disorders of the pulmonary system can also be treated according to the present invention. These disorders include, for example, asthma, chronic obstructive pulmonary disease (COPD), reactive airway disease, pulmonary fibrosis, and pulmonary hypertension.

Further disorders including an abnormal cellular proliferative component that can be treated according to the invention include Behcet's syndrome, fibrocystic breast disease, fibroadenoma, chronic fatigue syndrome, acute respiratory distress syndrome (ARDS), ischemic heart disease, post-dialysis syndrome, leukemia, acquired immune deficiency syndrome, vasculitis, lipid histiocytosis, septic shock, and familial intestinal polyposis such as Gardner syndrome. Also included in the scope of disorders that may be treated by the compositions and methods of the present invention are virus-induced hyperproliferative diseases including, for example, human papilloma virus-induced disease (e.g., lesions caused by human papilloma virus infection), Epstein-Barr virus-induced disease, scar formation, genital warts, cutaneous warts, and the like.

The pharmaceutical compositions of the present invention are further useful in the treatment of conditions and diseases of abnormal cell proliferation including various types of cancers such as primary tumors and tumor metastasis. Specific, non-limiting types of benign tumors that can be treated according to the present invention include hemangiomas, hepatocellular adenoma, cavernous hemangiomas, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, and pyogenic granulomas.

Representative, non-limiting cancers treatable according to the invention include breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

The pharmaceutical compositions of the present invention are also useful in preventing or treating proliferative responses associated with organ transplantation which contribute to rejections or other complications. For example, proliferative responses may occur during transplantation of the heart, lung, liver, kidney, and other body organs or organ systems.

B. Inflammation

The pharmaceutical compositions of the present invention are also useful in the treatment of diseases characterized by inflammation. Diseases and conditions which have significant inflammatory components are ubiquitous and include, for example, skin disorders, bowel disorders, certain degenerative neurological disorders, arthritis, autoimmune diseases and a variety of other illnesses. Some of these diseases have both an inflammatory and proliferative component, as described above. In particular embodiments the compounds are used to treat inflammatory bowel diseases (IBD), Crohn's disease (CD), ulcerative colitis (UC), chronic obstructive pulmonary disease (COPD), sarcoidosis, or psoriasis. The disclosed pharmaceutical compositions are also useful in the treatment of other inflammatory diseases, for example, allergic disorders, skin disorders, transplant rejection, poststreptococcal and autoimmune renal failure, septic shock, systemic inflammatory response syndrome (SIRS), adult respiratory distress syndrome (ARDS), envenomation, lupus erythematosus, Hashimoto's thyroiditis, autoimmune hemolytic anemias, insulin dependent diabetes mellitus, and rheumatic fever, pelvic inflammatory disease (PID), conjunctivitis, dermatitis, and bronchitis.

Inflammatory bowel diseases (IBD) includes several chronic inflammatory conditions, including Crohn's disease (CD) and ulcerative colitis (UC). Both CD and UC are considered “idiopathic” because their etiology is unknown. While Crohn's disease and ulcerative colitis share many symptoms (e.g., diarrhea, abdominal pain, fever, fatigue), ulcerative colitis is limited to the colon whereas Crohn's disease can involve any segment of the gastrointestinal tract. Both diseases may involve extraintestinal manifestations, including arthritis, diseases of the eye (e.g., episcleritis and iritis), skin diseases (e.g., erythema nodosum and pyoderma gangrenosum), urinary complications, gallstones, and anemia. Strokes, retinal thrombi, and pulmonary emboli are not uncommon, because many patients are in a hypercoagulable state.

In a particular embodiment, the pharmaceutical compositions of the present invention are useful in the treatment of inflammatory bowel disease. In a preferred embodiment, the inflammatory bowel disease is Crohn's disease.

Chronic Obstructive Pulmonary Disease, or COPD, is characterized by a not fully reversible airflow limitation which is progressive and associated with an abnormal inflammatory reaction of the lungs. It is one of the most common respiratory conditions of adults, a major cause of chronic morbidity and mortality, and represents a substantial economic and social burden worldwide (Pauwels R A. Lancet. (2004) 364(9434):613-20). Other names for the disorder include, for example, Chronic Obstructive Airways Disease, (COAD); Chronic Obstructive Lung Disease, (COLD), Chronic Airflow Limitation, (CAL or CAFL) and Chronic Airflow Obstruction (COA).

COPD is characterized by chronic inflammation throughout the airways, parenchyma, and pulmonary vasculature. The inflammation involves a multitude of cells, mediators, and inflammatory effects. Mediators include, for example, mediators include proteases, oxidants and toxic peptides. Over time, inflammation damages the lungs and leads to the pathologic changes characteristic of COPD. Manifestations of disease includes both chronic bronchitis and emphysema. Chronic bronchitis is a long-standing inflammation of the airways that produces a lot of mucus, causing wheezing and infections. It is considered chronic if a subject has coughing and mucus on a regular basis for at least three months a year and for two years in a row. Emphysema is a disease that destroys the alveolae and/or bronchae, causing the air sacs to become enlarged, thus making breathing difficult. Most common in COPD patients is the centrilobular form of emphysema. In a particular embodiment, the compositions of the present invention are useful in the treatment of chronic obstructive pulmonary disease.

Sarcoidosis is yet another chronic inflammatory disease with associated abnormal cell proliferation. Sarcoidosis is a multisystem granulomatous disorder wherein the granulomas are created by the angiogenic capillary sprouts providing a constant supply of inflammatory cells.

As noted above, inflammation also plays an important role in the pathogenesis of cardiovascular diseases, including restenosis, atherosclerotic complications resulting from plaque rupture, severe tissue ischemia, and heart failure. Inflammatory changes in the arterial wall, for example, are thought to play a major role in the development of restenosis and atherosclerosis (Ross R. N Engl J Med. (1999) 340: 115-126). Local inflammation occurs in the formation the plaques also contributes to the weakening of the fibrous cap of the advanced plaque, ultimately resulting in plaque rupture and acute coronary syndromes (Lind L. Atherosclerosis. (2003) 169(2):203-14).

Multiple sclerosis (MS) is a chronic, often debilitating autoimmune disease that affects the central nervous system. MS is characterized by inflammation which results when the body directs antibodies and white blood cells against proteins in the myelin sheath, fatty material which insulates the nerves in the brain and spinal cord. The result may be multiple areas of scarring (sclerosis), which slows or blocks muscle coordination, visual sensation and other nerve signals. In a particular embodiment, the pharmaceutical compositions of the present invention are useful in the treatment of multiple sclerosis.

Inflammatory have been shown to be associated with the pathogenesis of neurological disorders, including Parkinson's disease and Alzheimer's disease (Mirza B. et al. Neuroscience (2000) 95(2):425-32; Gupta A. Int J Clin Pract. (2003) 57(1):36-9; Ghatan E. et al. Neurosci Biobehav Rev. (1999) 23(5):615-33).

The present invention is also useful in the treatment of, for example, allergic disorders, allergic rhinitis, skin disorders, transplant rejection, poststreptococcal and autoimmune renal failure, septic shock, systemic inflammatory response syndrome (SIRS), adult respiratory distress syndrome (ARDS), envenomation, lupus erythematosus, myasthenia gravis, Grave's disease, Hashimoto's thyroiditis, autoimmune hemolytic anemias, insulin dependent diabetes mellitus, glomerulonephritis, and rheumatic fever, pelvic inflammatory disease (PID), conjunctivitis, dermatitis, bronchitis, and rhinitis.

C. Asthma

In particular embodiments the pharmaceutical compositions can be used in the treatment of asthma. In recent years, it has become clear that the primary underlying pathology of asthma is airway tissue inflammation (Lemanke (2002) Pediatrics 109(2):368-372; Nagayama et al. (1995) Pediatr Allergy Immunol. 6:204-208). Asthma is associated with numerous symptoms and signs (e.g., wheezing, cough, chest tightness, shortness of breath and sputum production). Airway inflammation is a key feature of asthma pathogenesis and its clinical manifestations. Inflammatory cells, including mast cells, eosinophils, and lymphocytes, are present even in the airways of young patients with mild asthma.

Inflammation also plays a role in wheezing disorders, with or without asthma. Asthma is sometimes classified by the triggers that may cause an asthma episode (or asthma attack) or the things that make asthma worse in certain individuals, such as occupational asthma, exercise induced asthma, nocturnal asthma, or steroid resistant asthma. Thus, the pharmaceutical compositions of the invention can also be used in the treatment of wheezing disorders, generally.

D. Arthritis and Osteoarthritis

More than 40 million Americans suffer from arthritis in its various forms, including includes over 100 kinds of rheumatic diseases (i.e., diseases affecting joints, muscle, and connective tissue, which makes up or supports various structures of the body, including tendons, cartilage, blood vessels, and internal organs). Representative types of arthritis include rheumatoid (such as soft-tissue rheumatism and non-articular rheumatism), fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropathies (ankylosing spondylitis), osteoarthritis, hyperuricemia and arthritis associated with acute gout, chronic gout, and systemic lupus erythematosus.

Hypertrophic arthritis or osteoarthritis is the most common form of arthritis and is characterized by the breakdown of the joint's cartilage. Osteoarthritis is common in people over 65, but may appear decades earlier. Breakdown of the cartilage causes bones to rub against each other, causing pain and loss of movement. In recent years, there has been increasing evidence that inflammation plays an important role in osteoarthritis. Nearly one-third of patients ready to undergo joint replacement surgery for osteoarthritis (OA) had severe inflammation in the synovial fluid that surrounds and protects the joints. In a particular embodiment, the pharmaceutical compositions of the present invention are useful in the treatment of osteoarthritis.

The second most common form of arthritis is rheumatoid arthritis. It is an autoimmune disease that can affect the whole body, causing weakness, fatigue, loss of appetite, and muscle pain. Typically, the age of onset is much earlier than osteoarthritis, between ages 20 and 50. Inflammation begins in the synovial lining and can spread to the entire joint. In another embodiment, the pharmaceutical compositions of the present invention are useful in the treatment of rheumatoid arthritis.

### EXPERIMENTAL

The present invention will now be described with specific reference to various examples. The following examples are not intended to be limiting of the invention and are rather provided as exemplary embodiments. As used in one or more examples below, “CH-1504” refers to a compound of formula (9), and such recitation may further define the compound as racemic or “DL” or as a purified enantiomer (i.e., the L-form or D-form). “MTX” refers to methotrexate.

### Example 1

### Salt Screening

The free acid form of the antifolate compound of Formula (9) has a crystalline structure but exhibits poor solubility. A salt screen of this compound was conducted with various pharmaceutically acceptable counterions to analyze aqueous solubility of the formed salts. The counterions used are provided in Table 1. Formed solids suspected of forming salts were analyzed by X-ray powder diffraction (XRPD).

TABLE 1

| Type of | | Type of | |
|---|---|---|---|
| Counterion | Counterion | Counterion | Counterion |
| Mineral acids | Sulfuric | Carboxylic | Benzoic |
| | Hydrochloric | acids | Citric |
| Sulfonic acids | Benzenesulfonic | | Fumaric |
| | 1,2-Ethandisulfonic | | Glycolic |
| | Ethanesulfonic | | Maleic |
| | Isethionic | | DL-malic |
| | Methansulfonic | | Oxalic |
| | 1,5-naphthalenedisulfonic | | Succinic |
| | 2-naphthalenesulfonic | | DL-tartaric |
| | toluenesulfonic | Bases | Ammonium |
| Amino acids | L-arginine | | Calcium |
| | L-lysine | | Potassium |
| | | | Sodium |

Of the various mineral, sulfonic, and carboxylic acids that were tested, crystalline salts were generated using HCl, benzenesulfonic acid, methansulfonic acid, 2-naphalenesulfonic acid, and ethanesulfonic acid. Salt formation was confirmed by ¹H NMR analysis. Solids exhibiting XRPD patterns of mostly amorphous material or with broad, low intensity peaks were obtained using 1,2-ethanedisulfonic acid, 1,5-naphthalenedisulfonic acid, sulfuric acid, and toluenesulfonic acid. No reaction was observed using benzoic acid, citric acid, glycolic acid, maleic acid, DL-malic acid, oxalic acid, fumaric acid, phosphoric acid, succinic acid, or DL-tartaric acid. The XRPD patterns of solids obtained using these acids were similar to the XRPD pattern of the crystalline acid compound of Formula (9).

Of the various bases that were tested, crystalline salts were generated using calcium methoxide. Solids exhibiting XRPD patterns of mostly amorphous material or with broad, low intensity peaks were obtained using ammonium hydroxide and potassium hydroxide. The XRPD pattern of solids obtained from a sodium salt exhibited one peak at about 5.0 2°θ. Salt attempts using L-arginine and L-lysine resulted in solids exhibiting XRPD patterns of mostly amorphous material or with broad peaks.

Hygroscopicity and approximate solubility in aqueous and buffered solutions of ammonium, besylate, calcium, esylate, sulfate, HCl, mesylate, napsylate, potassium, disodium, and tosylate salts were compared. In the hygroscopicity study, the salts were subjected to 75% relative humidity for five days. A new form was obtained from the calcium salt. The ammonium, besylate, esylate, HCl, mesylate, and napsylate salts remained unchanged, but peak shifting was observed with the ammonium and napsylate salts. Tacky or gummy solids or solids not exhibiting birefringence and extinction were obtained from the amorphous sulfate, potassium, disodium, and tosylate salts.

The salts were screened for aqueous solubility as well as solubility in pH 5, 6, and 7 buffer solutions. The solubilities were estimated based on visual observation and do not necessarily reflect the equilibrium solubility. In some samples, when solids remained, the slurry was checked after 1 and 2 days to determine dissolution. The disodium salt exhibited an approximate aqueous solubility of >116 mg/mL, and the potassium salt exhibited an approximate solubility of >98 mg/mL. The remaining salts exhibited an approximate aqueous solubility of 0.4 mg/mL or less.

When tested in a pH 7 (20 mM phosphate) buffer solution, solubility trends were similar to those observed in water. The disodium and dipotassium salts demonstrated the highest solubility (≧32 mg/mL and ≧16 mg/mL, respectively). Solubility of the napsylate salt was ≧1.1 mg/mL, and besylate solubility was ≧2.0 mg/mL. All other salts investigated showed solubilities of <0.2 mg/mL.

Based on the above data, the besylate, napsylate, potassium, and sodium salts were tested in further solubility studies. Approximate solubilities in solutions of pH 5 and 6 were determined. Solubilities were also determined in a pH 7 buffer with increased buffering capacity. Both the besylate and napsylate salts demonstrated a solubility of 0.4 mg/mL at all pH ranges. The disodium salt solubility was ≧37 mg/mL at pH 7 and ≧40 mg/mL at pH 5 and 6. The solubility of the dipotassium salt, measured at pH 7, was ≧16 mg/mL.

The disodium and dipotassium salts were prepared on a larger scale and crystallized in water/IPA and water/acetone. The crystalline disodium salt of the compound of Formula (11), which is designated as Form A (Na), was obtained from both solvent systems. The poorly crystalline dipotassium salt of the compound of Formula (11), which is designated as Form A (K), was obtained from water/IPA. Solids obtained from water/acetone showed slightly improved crystallinity, but the solids still were poorly crystalline.

An abbreviated polymorph screen of the disodium salt of the compound of Formula (9) was conducted, and two crystalline forms were isolated and characterized (designated forms A and B). An amorphous form was also generated. Disodium salt Form A was a crystalline, non-hygroscopic solid containing approximately 4.5 moles of water per mole of the disodium salt of the compound of Formula (11). As described above, disodium salt Form A was a crystalline solid obtained using a water/IPA system or a water/acetone system. Karl Fischer analysis confirmed a water content of 14.8% (equivalent to about 4.75 moles of water per one mole of disodium salt). Hygroscopicity studies showed the material was non-hygroscopic, as determined by visual assessment, when stored at 58% and 75% relative humidity for 14 days, though the XRPD pattern indicated a reduction in crystallinity after storage in 75% RH. VT-XRPD indicated the material lost crystallinity upon heating to 70° C. under a purge of nitrogen. Heating was continued to achieve a temperature of 90° C. Crystallinity was not regained upon cooling to ambient.

Disodium salt Form B was a crystalline hexahydrate obtained from fast evaporation using methanol and trifluoroethanol. Karl Fischer analysis showed 17.5% water (about 6 moles).

The X-ray powder diffraction pattern graph (Cu Kα radiation) of the racemic, disodium salt of the compound of Formula (11)—disodium salt Form A from above—is illustrated in FIG. 5, which shows signal intensity at 2°θ. The interplanar spacing peaks of specific 2°θ angles, absolute peak heights, D-spacing, and peak relative intensities of various peaks illustrated in FIG. 5 are provided below in Table 2.

TABLE 2

| Position (2° θ) | Height (Cts) | D-Spacing (A) | Retative Intensity (%) |
|---|---|---|---|
| 4.8750 | 449.49 | 18.10095 | 16.28 |
| 7.3490 | 472.36 | 12.01931 | 17.11 |
| 8.1221 | 2314.59 | 10.87699 | 83.85 |
| 10.5019 | 1101.18 | 8.41690 | 39.89 |
| 11.8701 | 279.44 | 7.44962 | 10.12 |
| 12.4449 | 1386.78 | 7.10681 | 50.24 |
| 14.5270 | 2760.27 | 6.09255 | 100.00 |
| 16.0326 | 1516.46 | 5.52364 | 54.94 |
| 17.1551 | 111.38 | 5.16466 | 40.26 |
| 20.6738 | 2337.29 | 4.29288 | 84.68 |
| 21.1909 | 1587.11 | 4.18930 | 57.50 |
| 21.7468 | 1392.27 | 4.08345 | 50.44 |
| 22.5306 | 777.83 | 3.94315 | 28.18 |
| 23.2841 | 530.22 | 3.81721 | 19.21 |
| 23.9665 | 2401.93 | 3.71003 | 87.02 |
| 24.4918 | 1100.70 | 3.63165 | 39.88 |
| 28.3375 | 349.14 | 3.14692 | 12.65 |
| 29.1428 | 1094.89 | 3.06177 | 39.67 |
| 30.8958 | 359.50 | 2.89192 | 13.02 |
| 32.2118 | 487.65 | 2.77672 | 17.34 |
| 33.5960 | 294.64 | 2.66541 | 10.67 |
| 34.5266 | 355.79 | 2.59567 | 12.89 |
| 35.4153 | 273.34 | 2.53254 | 9.90 |

### Examples 2-8

### Improvements in Pharmacokinetics Using Inventive Formulation

The pharmacokinetic parameters of a single oral dose of the antifolate compound according to the invention were evaluated. In Comparative Examples 2-7, 1 to 20 mg of an antifolate compound according to Formula (9) was administered in the racemic free acid form (i.e., not as part of a pharmaceutical formulation). The drug product was supplied as powder-filled gelatin capsules in three active strengths (1.0 mg, 2.5 mg. and 5.0 mg) with each capsule including enough microcrystalline cellulose to bring the total capsule weight to 288 mg. In Example 8 (the inventive formulation), only 1 mg of an antifolate compound according to Formula (11) (the racemic disodium salt) was administered as a pharmaceutical formulation according to the invention comprising GELUCIRE® 44/14, mannitol, magnesium stearate, and colloidal silica. In Examples 2-8, the test material was administered to a healthy male subject, and blood samples were taken before dosing and at 0.5, 1, 1.5, 2, 2.5, 3, 3.5, 4, 5, 6, 8, 10, 12, 16, 24, and 48 hours after dosing. The calculated pharmacokinetic values observed are provided below in Table 3.

TABLE 3

| | Antifolate Dose | Cₘₐₓ | tₘₐₓ | AUC₀₋ₜ | AUC_{0-∞} | t_{1/2} |
|---|---|---|---|---|---|---|
| Example | (mg) | (ng/mL) | (hours) | (ng · h/mL) | (ng · h/mL) | (hours) |
| 2 (comparative) | 1 | 0.69 | 2.26 | 1.25 | 1.52 | 0.99 |
| 3 (comparative) | 5 | 2.65 | 1.26 | 8.92 | 9.58 | 3.21 |
| 4 (comparative) | 7.5 | 2.05 | 1.50 | 6.63 | 7.47 | 2.71 |
| 5 (comparative) | 10 | 6.00 | 2.01 | 25.2 | 26.0 | 3.13 |
| 6 (comparative) | 15 | 6.57 | 2.25 | 25.0 | 25.9 | 3.20 |
| 7 (comparative) | 20 | 7.83 | 2.25 | 34.6 | 35.6 | 3.91 |
| 8 (inventive) | 1 | 9.05 | 1.00 | 23.98 | 24.55 | 2.39 |

In Table 3, Cₘₐₓ is the maximum measured plasma concentration of the antifolate compound administered and tₘₐₓ is the time to Cₘₐₓ. As seen above, administration of 1 mg of the antifolate compound alone in the free acid form resulted in a Cₘₐₓ of only 0.69 ng/mL, but administration of 1 mg of the antifolate compound in the disodium salt form as part of the inventive pharmaceutical composition resulted in a Cₘₐₓ of 9.05, which is a more than 13-fold increase in Cₘₐₓ. Moreover, administration of 1 mg of the inventive antifolate disodium salt pharmaceutical composition (Example 8) resulted in a greater Cₘₐₓ than when administering 20 times the amount of the diacid antifolate compound alone (Example 7). Thus, the pharmaceutical formulations of the present invention allow for greatly reducing the amount of antifolate compound that is administered to a subject while actually increasing the amount of the compound that is available for therapeutic action. Additionally, as seen in Table 3, administering the antifolate compound as part of the inventive composition reduces tₘₐₓ.

### Example 9

### Pharmaceutical Composition and Method of Preparation Thereof

Mannitol and colloidal silicon dioxide were blended in a high shear granulator bowl to form a homogenous blend. GELUCIRE© 44/14 was divided into two portions for use in forming the composition (i.e., the “dispersion portion” and the “rinse portion”). The dispersion portion of the GELUCIRE© 44/14 was heated to approximately 60° C. and then reduced to approximately 50° C. The drug component (a 4.5 hydrate of a disodium salt according to Formula (11)) was slowly added to the GELUCIRE© 44/14 while homogenizing (for example, with a Polytron Homogenizer (model PT 10/35)). Once the entire content of the drug was added and dispersed into the GELUCIRE© matrix, the molten mixture was added to the granulated mixture of mannitol and colloidal silicon dioxide while blending.

The rinse portion of the GELUCIRE© 44/14 was heated to approximately 60° C. and added to the container that contained the active pharmaceutical ingredient (API) and the GELUCIRE© 44/14 to rinse-off any API remaining in the container. This rinse portion was then added to the granulator bowl while blending to form a mixture of the drug component, the full content of GELUCIRE© 44/14, mannitol, and colloidal silicon dioxide. The contents of the granulator bowl were discharged, wet screened, and allowed to dry at room temperature.

After drying was completed, the dried granulation material was screened. The screened material was then blended with additional (“extra-granular”) colloidal silicon dioxide, additional (“extra-granular”) mannitol, and magnesium stearate in a V-Blender. The blend was encapsulated into hard gelatin capsules using an In-Cap encapsulation machine (available from Dott. BONAPACE & C., Milan, Italy). The components of the prepared composition are provided below in Table 4.

TABLE 4

| | Quantity |
|---|---|
| Component | (mg/g) |
| Drug component | 6.41 |
| Mannitol PEARLITOL © 100 SD Roquette (intragranular) | 563.00 |
| Mannitol PEARLITOL © 100 SD Roquette (extragranular) | 319.00 |
| GELUCIRE © 44/14 (dispersion portion) | 60.00 |
| GELUCIRE © 44/14 (rinse portion) | 33.59 |
| Colloidal silicon dioxide USP/EP (intragranular) | 5.00 |
| Colloidal silicon dioxide USP/EP (extragranular) | 5.00 |
| Magnesium stearate NF/EP non-bovine (#5712) | 8.00 |
| Total: | 1000.00 |

### Example 10

### Pharmaceutical Composition and Method of Preparation Thereof

Mannitol, Cyclodextrin (CAVAMAX© W7, available from Wacker Chemie, AG), and the drug component (a 4.5 hydrate of the disodium salt according to Formula (11)) were bag-blended, and screened through an 80 mesh screen (approximately 180 microns) into a high shear granulator bowl. The remaining mannitol was hand screened into the granulator bowl. The contents of the high shear granulator bowl were blended, and colloidal silicon dioxide was added followed by further blending. The magnesium stearate then added followed by further blending. The blend was encapsulated into hard gelatin capsules using an In-Cap encapsulation machine. The components of the prepared composition are provided below in Table 5.

TABLE 5

| | Quantity |
|---|---|
| Component | (mg/g) |
| Drug component | 6.41 |
| Mannitol PEARLITOL © 100 SD Roquette (first portion) | 100.00 |
| Mannitol PEARLITOL © 100 SD Roquette | 785.00 |
| CAVAMAX © W7 β-cyclodextrin | 93.60 |
| Colloidal silicon dioxide USP/EP | 5.00 |
| Magnesium stearate NF/EP non-bovine (#5712) | 10.00 |
| Total: | 1000.00 |

### Example 11

### [³H]MTX Transport Inhibition

Transport of 2 μM [³H]MTX (methotrexate) at 37° by intact CCRF-CEM human T-cell leukemia was assayed by a micro-method utilizing repeated iced saline washes to remove extracellular drug. Such method is disclosed in McGuire J J, et al., Cancer Res 1989; 49:4517-25 and McGuire J J, et al., Cancer Res 2006; 66:3836-44, both of which are incorporated herein by reference in their entirety. The washed cell pellets were solubilized in 1 ml of 0.3% Triton X-100 at 37° C. for 1 hour before transfer to scintillation vials; 10 ml Ecoscint liquid scintillation fluid (National Diagnostics, Atlanta, Ga.) was added and radioactivity was quantitated in a Beckman LS6500 scintillation counter. Intracellular radiolabel was analyzed by HPLC and was shown to be at least 79%, and typically >90%, MTX. Inhibitory potency of analogs was assessed by pre-mixing [³H]MTX with five graded concentrations of analog in 50 μl, such that when diluted to 250 μL with cells the final [³H]MTX concentration was 2 μM (2 μCi/ml) and the compound concentration was as required. Uptake was initiated by addition of 200 μL of cells at ≈2.5×107 cells/ml and 2 aliquots (100 μL) were removed to iced saline and processed at 5 min. Adventitious [³H]MTX binding was determined at 0° C. by adding 200 μl of cells to 25 μl of PBS in a tube and cooling to 0° C. in ice for ≧5 min; following addition of 25 μl of [³H]MTX to achieve a final concentration of 2 μM, 2 aliquots (100 μL) were immediately removed to iced saline and processed. Controls within each experiment showed that [³H]MTX uptake in the absence of analog was linear for 5 min under these conditions; control uptake was typically 12 μmol/107 cells/5 min. IC50 values were determined and are illustrated below in Table 6.

Analytical HPLC was performed on a Rainin Instruments HPLC system using the Dynamax controller and data capture module run on a Macintosh computer, such as described in McGuire J J, et al., J Biol Chem 1990; 265:14073-9, which is incorporated herein by reference in its entirety. C18 reversed-phase (0.4×25 cm; Rainin Microsorb, 5μ) HPLC was performed at 25° C. Detection was by absorbance at 280 and/or 254 nm. For MTX (tr, ≈31.6 min) and 7-OH-MTX (tr, ≈35.2 min) the gradient was from 4-13% ACN in 0.1 M Na-acetate, pH 5.5 over 41 min at 1 ml/min. Compounds did not elute under these conditions; the gradient was adjusted to 4-20% ACN in 0.1 M Na-acetate, pH 5.5 over 41 min.

TABLE 6

| | [³H]MTX transport |
|---|---|
| Compound | inhibition (IC₅₀) (μM) |
| Aminopterin | 1.5 |
| D-MTX | 49 |
| DL-CH-1504 | 1.7 |
| L-CH-1504 | 1.1 |
| D-CH-1504 | 7.6 |

As illustrated in Table 6, the enantiomerically pure form of CH-1504 (L-CH-1504) was shown to be more efficiently transported into cells expressing the reduced folate carrier (RFC) in comparison to the other compounds tested.

### Example 12

### Cell Culture and Growth Inhibition

The human T-lymphoblastic leukemia cell line CCRF-CEM (described in Foley G F, et al., Cancer 1965; 18:522-9) was cultured as described in McCloskey D E, et al., J Biol Chem 1991; 266:6181-7 (both of which are incorporated herein by reference in their entirety) and verified to be negative for Mycoplasma contamination (Mycoplasma Plus PCR primers, Stratagene, La Jolla, Calif.). Growth inhibition of CCRF-CEM cells by continuous (120 hr) drug exposure was assayed as described in Foley and in McGuire J J, et al., Oncology Res 1997; 9:139-47. EC50 values (drug concentration effective at inhibiting cell growth by 50%) were interpolated from plots of percent growth relative to a solvent-treated control culture versus the logarithm of drug concentration by performing a linear regression of the two data points on either side of 50% relative growth and calculating the inhibitor concentration corresponding to 50% relative growth. Results are provided in Table 7.

TABLE 7

| | Growth Inhibition |
|---|---|
| Compounds | (EC₅₀) (nM) |
| MTX | 15 |
| DL-CH-1504 | 8.6 |
| L-CH-1504 | 6.1 |
| D-CH-1504 | 29 |

As illustrated in Table 7, the L-form of CH-1504 exhibits greater growth inhibition as compared to the D-form or the racemic form.

### Example 13

### Plasma Concentration

Racemic CH-1504 was administered once orally to fasted female Lewis rats at a dose of 10 mg/kg (vehicle: 0.11% carboxymethylcellulose/0.45%) TWEEN 80, formulation: suspension). About 750 μL of blood was collected from the jugular vein at 1 and 3 hours after administration. And then, whole of blood was collected from the femoral vein under diethyl ether anesthesia at 6 hours after administration. The collected blood was immediately centrifuged to obtain a plasma sample. L- and D-CH-1504 were extracted from the plasma by solid-phase extraction and were then determined with a LC/MS/MS. Plasma concentrations of L- and D-CH-1504 at each sample are shown in Table 8. Plasma concentrations of L- and D-CH-1504 were not equivalent, showing a difference in pharmacokinetic parameters of each enantiomer. In particular, as illustrated in Table 8, the L-form of CH-1504 exhibited significantly higher plasma concentrations at every collection interval as compared to the D-form, clearly indicating higher bioavailability.

TABLE 8

| | | | | Plasma conc. | |
|---|---|---|---|---|---|
| | | | Time after | (ng/mL) | |
| | Dose | Animal | Administration | L-CH- | D-CH- |
| Compound | (mg/kg) | No. | (h) | 1504 | 1504 |
| Racemic | 10 | YF01 | 1 | 10.6 | 3.12 |
| CH-1504 | | | 3 | 9.82 | 6.79 |
| | | | 6 | 8.53 | 3.91 |
| | | YF02 | 1 | 3.16 | 0.904 |
| | | | 3 | 1.77 | 1.09 |
| | | | 6 | 1.67 | 1.71 |
| | | YF03 | 1 | 3.60 | 1.36 |
| | | | 3 | 5.34 | 32.6 |
| | | | 6 | 10.0 | 5.69 |

### Example 14

### Plasma Concentration

L- or D-CH-1504 was administered once orally to non-fasted female Lewis rats at a dose of 10 mg/kg (vehicle: 0.11% carboxymethylcellulose/0.45% TWEEN 80, formulation: suspension). About 750 μL of blood was collected from the jugular vein at 1 and 3 hours after administration. And then, whole of blood was collected from the femoral vein under diethyl ether anesthesia at 6 hours after administration. The collected blood was immediately centrifuged to obtain a plasma sample. L- and D-CH-1504 were extracted from the plasma by solid-phase extraction and were then determined with a LC/MS/MS. Plasma concentrations of L- and D-CH-1504 at each sample are shown in Table 9. In all samples, isomerization of CH-1504 could not be confirmed by 6 hours after administration of each enantiomer. These results again illustrate significantly higher plasma concentrations for the L-form of the drug.

TABLE 9

| | | | | Plasma conc. | |
|---|---|---|---|---|---|
| | | | Time after | (ng/mL) | |
| | Dose | Animal | Administration | L-CH- | D-CH- |
| Compound | (mg/kg) | No. | (h) | 1504 | 1504 |
| L-CH-1504 | 10 | YF11 | 1 | 118 | BLQ |
| | | | 3 | 59.7 | BLQ |
| | | | 6 | 21.7 | BLQ |
| | | YF12 | 1 | 144 | BLQ |
| | | | 3 | 61.9 | BLQ |
| | | | 6 | 22.7 | BLQ |
| | | YF13 | 1 | 139 | BLQ |
| | | | 3 | 36.8 | BLQ |
| | | | 6 | 22.2 | BLQ |
| D-CH-1504 | 10 | YF21 | 1 | 0.895 | 31.5 |
| | | | 3 | BLQ | 14.3 |
| | | | 6 | BLQ | 8.34 |
| | | YF22 | 1 | BLQ | 20.5 |
| | | | 3 | BLQ | 9.44 |
| | | | 6 | BLQ | 13.6 |
| | | YF23 | 1 | BLQ | 11.0 |
| | | | 3 | BLQ | 8.93 |
| | | | 6 | BLQ | 8.01 |

BLQ: Below limit of quantification (<0.500 ng/mL)

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A pharmaceutical composition comprising an antifolate compound according to Formula (6): 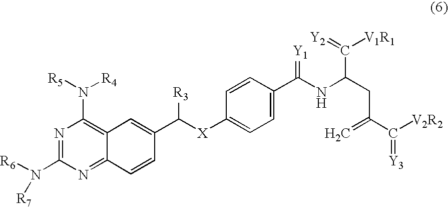
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
and further comprising an excipient that increases one or both of solubility and bioavailability of the antifolate compound, the excipient being selected from the group consisting of cyclodextrins, polyglycolized glycerides, and combinations thereof.

2. The pharmaceutical composition according to claim 1, wherein the excipient comprises a polyglycolized glyceride.

3. The pharmaceutical composition according to claim 2, wherein the polyglycolized glyceride has a melting point of less than about 50° C.

4. The pharmaceutical composition according to claim 2, wherein the polyglycolized glyceride has an HLB value that is greater than about 8.

5. The pharmaceutical composition according to claim 2, wherein the polyglycolized glyceride comprises a C₁₄-C₂₀ fatty acid ester.

6. The pharmaceutical composition according to claim 5, wherein the fatty acid ester is a glyceryl ester.

7. The pharmaceutical composition according to claim 2, wherein the polyglycolized glyceride comprises a polyethylene glycol ester having a number average MW of about 1,200 to about 2,500 Da.

8. The pharmaceutical composition according to claim 2, wherein the polyglycolized glyceride is a PEG1500 ester of glyceryl laurate having a melting point of 44° C. and an HLB of 14.

9. The pharmaceutical composition according to claim 2, wherein the polyglycolized glyceride and the antifolate compound are present at a ratio of about 1:1 to about 50:1.

10. The pharmaceutical composition according to claim 1, wherein the excipient comprises a cyclodextrin.

11. The pharmaceutical composition according to claim 1, wherein the antifolate compound comprises a compound according to formula (7): 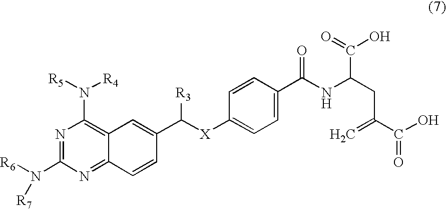
wherein:
X is CHR₈ or NR₈;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof.

12. The pharmaceutical composition according to claim 1, wherein the antifolate compound comprises a compound according to Formula (9): 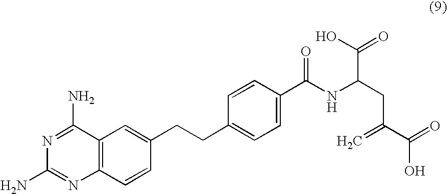
or a pharmaceutically acceptable ester, amide, salt, solvate, enantiomer, or prodrug thereof.

13. The pharmaceutical composition according to claim 1, wherein the antifolate compound comprises a compound according to Formula (11): 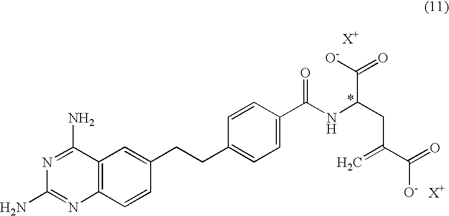
or an enantiomer thereof, wherein each X⁺ independently is a salt-forming counterion.

14. The pharmaceutical composition according to claim 13, wherein X⁺ is an alkali metal cation.

15. The pharmaceutical composition according to claim 13, wherein X⁺ is sodium.

16. The pharmaceutical composition according to claim 13, wherein X⁺ is potassium.

17. The pharmaceutical composition according to claim 13, wherein the antifolate compound is a crystalline salt.

18. The pharmaceutical composition according to claim 13, wherein the antifolate compound is a racemic salt.

19. The pharmaceutical composition according to claim 13, wherein the antifolate compound comprises a compound according to Formula (12): 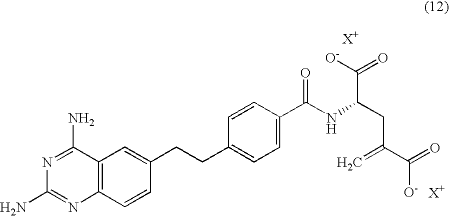
wherein each X⁺ independently is a salt-forming counterion, and wherein the antifolate compound is in the (S) enantiomeric form.

20. The pharmaceutical composition according to claim 19, wherein the antifolate compound exhibits an enantiomeric purity for the (S) enantiomer of at least about 90%.

21. The pharmaceutical composition according to claim 19, wherein the antifolate compound exhibits an enantiomeric purity for the (S) enantiomer of at least about 95%.

22. The pharmaceutical composition according to claim 19, wherein the antifolate compound exhibits an enantiomeric purity for the (S) enantiomer of at least about 99%.

23. The pharmaceutical composition according to claim 19, wherein the antifolate compound comprises a compound according to Formula (12) that is a crystalline, disodium salt in the (S) enantiomeric form exhibiting an enantiomeric purity for the (S) enantiomer of at least about 99%.

24. The pharmaceutical composition according to claim 19, wherein the antifolate compound comprises a compound according to Formula (12) that is a crystalline, dipotassium salt in the (S) enantiomeric form exhibiting an enantiomeric purity for the (S) enantiomer of at least about 99%.

25. The pharmaceutical composition according to claim 1, further comprising a bulking agent.

26. The pharmaceutical composition according to claim 25, wherein the bulking agent comprises mannitol.

27. The pharmaceutical composition according to claim 1, further comprising a lubricant.

28. The pharmaceutical composition according to claim 27, wherein the lubricant comprises magnesium stearate.

29. The pharmaceutical composition according to claim 1, further comprising an anti-adherent.

30. The pharmaceutical composition according to claim 28, wherein the anti-adherent comprises silicon dioxide.

31. The pharmaceutical composition according to claim 1, wherein the composition further comprises mannitol, magnesium stearate, and silicon dioxide.

32. A method for treating a condition selected from the group consisting of abnormal cell proliferation, inflammation, asthma, and arthritis, said method comprising administering to a subject in need of treatment a pharmaceutical composition according to claim 1.

33. A pharmaceutical composition comprising an alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%;
and further comprising an excipient that increases one or both of solubility and bioavailability of the alkali metal salt compound.

34. The pharmaceutical composition according to claim 33, wherein the excipient comprises fatty acid esters of glycerol and polyethylene glycol esters.

35. The pharmaceutical composition according to claim 33, wherein the excipient comprises a cyclodextrin.

36. The pharmaceutical composition according to claim 33, wherein the salt is in a stable, crystalline form.

37. A method of making a pharmaceutical composition comprising an antifolate compound according to Formula (6): 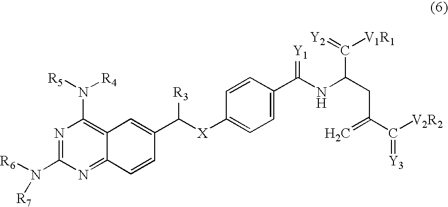
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₉ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
the method comprising:
forming a mixture of the antifolate compound, a molten polyglycolized glyceride, a first amount of a bulking agent, and a first amount of a lubricant;
granulating the formed mixture; and
combining the granulated mixture with a second amount of a bulking agent and a second amount of a lubricant.

38. The method according to claim 37, wherein the antifolate compound comprises a compound according to Formula (12): 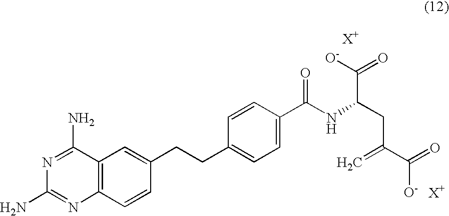
wherein each X⁺ independently is a salt-forming counterion, and wherein the antifolate compound is in the (S) enantiomeric form.

39. The method according to claim 38, wherein the antifolate compound exhibits an enantiomeric purity for the (S) enantiomer of at least about 90%.
